(19) United States
(12) Patent Application Publication
(10) Pub. No.: US 2025/0144035 A1
(43) Pub. Date: May 8, 2025

(54) Method of Increasing Epithelial Permeability Using Nanoparticles

(71) Applicant: Carnegie Mellon University, Pittsburgh, PA (US)

(72) Inventors: Kathryn Ann Whitehead, Pittsburgh, PA (US); Nicholas George Lamson, LaFayette, NY (US)

(21) Appl. No.: 19/015,088

(22) Filed: Jan. 9, 2025

Related U.S. Application Data
(63) Continuation of application No. 16/630,219, filed on Jan. 10, 2020, now abandoned.
(60) Provisional application No. 62/604,636, filed on Jul. 13, 2017.
(60) Provisional application No. 62/763,204, filed on Jun. 6, 2018.

(51) Int. Cl. A61K 9/51; A61K 9/00; A61K 38/26; A61K 38/28; A61K 45/06; A61P 3/10

(52) U.S. Cl. CPC A61K 9/5115; A61K 9/006; A61K 38/26; A61K 38/28; A61K 45/06; A61P 3/10

(57) ABSTRACT

Provided herein are devices and dosage forms useful in delivering macromolecular active ingredients or drugs, such as proteins, peptides and nucleic acids, through epithelial membranes, such as intestinal epithelium. Also provided are trans-epithelial drug delivery methods and methods of treatment of diabetes or insulin resistance, or to induce weight loss.

### CROSS REFERENCE TO RELATED APPLICATIONS

This application is a continuation of U.S. patent application Ser. No. 16/630,219, filed Jul. 13, 2018, which is the United States national phase of International Application No. PCT/US2018/042035 filed Jul. 13, 2018, which claims the benefit of U.S. Provisional Patent Application Nos. 62/604,636 filed Jul. 13, 2017, and 62/763,204 filed Jun. 6, 2018, the disclosures of which are hereby incorporated by reference in their entireties.

### REFERENCE TO A SEQUENCE LISTING

The Sequence Listing associated with this application is filed in electronic format via EFS-Web and is hereby incorporated by reference into the specification in its entirety. The name of the computer-readable file containing the Sequence Listing is 2409042.xml. The size of the file is 3,510 bytes, and the file was created on Jan. 3, 2025.

### BACKGROUND

Provided herein are methods and compositions for increasing the permeability of epithelial barriers using nanoparticles.

A challenge in delivering macromolecules (e.g., proteins and nucleic acids) to patients is a lack of permeability of epithelial tissue. For example, oral delivery of macromolecules is hampered by the lack of permeability of the intestinal lining, resulting from both the presence of mucus and the epithelial cellular barrier. One approach to improving permeability is the use of chemical permeation enhancers, which include any compounds that promote transport across a mass transfer barrier. Previous studies have shown that certain synthetic small molecules and polymer chains can greatly increase the permeability of epithelial layers, such as those that line the intestines, allowing better absorption of macromolecule drugs. In particular, macromolecular permeability can be enhanced through modulation of the tight junctions, which are dynamic protein structures that connect epithelial cells and form a diffusion barrier between them. Unfortunately, nearly all previously-reported permeation enhancers, which include detergents, acids, salts, and nitrogenous small molecules, are associated with corresponding toxicity or immunogenicity. This issue has prevented permeation enhancers as a whole from being effectively implemented in clinical delivery applications.

In another approach, macromolecules are loaded into or covalently bonded onto nanoparticles, with the intention of the entire complex being taken up across the intestinal barrier. Nevertheless, effective delivery approaches for macromolecules, such as biologics or drugs, across epithelial tissue, such as intestinal epithelium, are lacking. Non-parenteral dosage forms for delivery of insulin or other biologics or drugs, such as exenatide, also are needed for effective treatment of diabetes.

### SUMMARY

In one aspect, a dosage form is provided. The dosage form comprises negatively-charged nanoparticles having an average diameter, e.g., a Z average diameter as determined by dynamic light scattering, of less than 1 μm, 500 nm, less than 200 nm, or less than 100 nm, e.g., 50 nm or 20 nm, an active ingredient of less than 40 kDa, 25 kDa, or 10 kDa, or a hydrodynamic radius of 10 nm or less, and a pharmaceutically acceptable excipient.

In another aspect, a trans-epithelial drug delivery method is provided. The method comprises: contacting an epithelial membrane with a negatively-charged nanoparticle having an average diameter, e.g., a Z average diameter as determined by dynamic light scattering, of less than 1 μm, 500 nm, less than 200 nm, or less than 100 nm, e.g., 50 nm or 20 nm; and then contacting the epithelial membrane with an active ingredient of less than 40 kDa, 25 kDa or less, or 10 kDa or less, or a hydrodynamic radius of 10 nm or less.

In another aspect, a method of treating diabetes, e.g., type 1 or type 2 diabetes, or insulin resistance, or inducing weight loss, in a patient is provided. The method comprises administering to the patient an amount of insulin or a glucagon-like peptide-1 receptor agonist, such as exenatide, liraglutide; lixisenatide; albiglutide; dulaglutide; semaglutide; or taspoglutide, effective to treat diabetes or insulin resistance or to induce weight loss in a patient, thereby treating the diabetes or insulin resistance, or inducing weight loss, in the patient.

### BRIEF DESCRIPTION OF THE DRAWINGS

FIGS. 1A and 1B depict schematically versions of a mucoadhesive patch as described herein.

FIGS. 2A-2C depict schematically versions of a mucoadhesive patch as described herein.

FIGS. 3A and 3B depict schematically versions of a mucoadhesive patch as described herein.

FIG. 4. Nanoparticles do not induce cytotoxicity in Caco-2 cells. Nanoparticles were examined at a concentration of 0.2% w/v using the MTT cell viability assays. None of the treatments induced statistically significant reductions in viability at p<0.05. Error bars display s.e.m. (n=4).

FIGS. 5A-5C. Silica nanoparticles increase Caco-2 monolayer permeability more potently with decreasing size. The particle treatments were applied at a concentration of 0.2 wt. %, and the measurements are each expressed as ratios to untreated control wells (dotted black lines). (FIG. 5A) Particles less than 200 nm in diameter significantly reduced TEER, with smaller particles causing greater increases in permeability. This trend was reflected in transepithelial passage of the permeation markers (FIG. 5B) calcein and (FIG. 5C) 4 kDa FITC-Dextran, which were both improved to greater extents by smaller particles. Error bars display s.e.m. (n=3), *p<0.05 0.05 with respect to control wells.

FIGS. 6A and 6B. Permeation enhancement of Caco-2 monolayers by bare silica nanoparticles is dose dependent. Epithelia treated with both (FIG. 6A) 50 nm particles and (FIG. 6B) 20 nm particles both showed a clear trend of increasing calcein permeability (with respect to untreated control wells, denoted by dotted black line) due to increasing particle concentrations. Error bars display s.e.m. (n=3), *p<0.05 with respect to control wells.

FIGS. 7A and 7B. Nanoparticles with negatively charged surface chemistries increase Caco-2 monolayer permeability, while neutral and positively charged particles show little activity. All particles were nominally 50 nm in diameter, tested at a concentration of 0.2 wt. %, and normalized to untreated control wells (dotted black lines). (FIG. 7A) The greater ability of particles with negatively charged surfaces to reduce TEER was reflected in (FIG. 7B) the greater passage of calcein through monolayers treated with negative particles. Error bars display s.e.m. (n=3), *p<0.05 with respect to control wells.

FIG. 8. Oral treatment with silica particles improved absorption of FITC-labelled, 4 kDa dextran (FITC-DX4). 50 nm particles were the most effective enhancers, followed by 100 nm. Despite their supreme efficacy in cell culture models of the intestines, 20 nm silica particles were not effective in mice. Error bars display s.e.m. (n=6). *p<0.05 by one-tailed t-test, with respect to PBS control mice.

FIG. 9. 20 nm particles show poor transport through in vitro intestinal mucus when compared with 50 nm particles. 2 mm of simulated intestinal mucus (5% Type II Mucin in PBS) was placed onto permeable transwell membranes. Fluorescent particles were added on top of the mucus layer, and the basal particle concentration was sampled over time for particle concentration. Transport of the smaller, Rhodamine B labelled particles was impeded to a greater extent than that of the larger, FITC labelled particles. Error bars display s.e.m. (n=3).

FIGS. 10A-10C. 20 nm silica particles bind to intestinal mucus, while larger particles do not. 20 nm particles increased drastically in size within 30 minutes of exposure to a Type II mucin solution (FIG. 10A). By contrast, (FIG. 10B) 50 nm and (FIG. 10C) 100 nm silica particles did not bind to mucin or change size in its presence.

FIG. 11. Negatively charged particles improved absorption of FITC-DX4 in mice, while positive and neutral particles did not. Among a collection of 50 nm particles, larger increases in FITC-DX4 uptake generally correlated with more negative surface charge, with silica particles being the most effective. Error bars display s.e.m. (n=6). *p<0.05 by one-tailed t-test, with respect to PBS control mice.

FIG. 12. Silica nanoparticles enabled intestinal insulin delivery in mice. Two hours following oral gavage of silica particles to mice, an intestinal injection of 1 U/kg insulin induced sustained reductions in blood glucose levels. Polystyrene nanoparticles did not enable insulin absorption, while a 1 U/kg subcutaneous insulin dose induced a pronounced, but brief response. Integrated areas above the blood glucose curves show that oral delivery with silica nanoparticles and subcutaneous injection resulted in comparable total pharmacodynamic effect. Error bars display s.e.m. (n=5). *p<0.05 by one-tailed t-test.

FIG. 13. Particle-assisted, intestinal insulin delivery was dose responsive to quantity of particles given. At a constant insulin dose of 1 U/kg, increasing particle doses caused more drastic and sustained reductions in blood glucose, which were reflected in the integrated areas above the curves. Error bars display s.e.m. (n=5). #p<0.05 by one-way ANOVA.

FIG. 14. Particle-assisted, intestinal insulin delivery was dose responsive to quantity of insulin given. At a constant particle dose of 100 mg/kg, increasing insulin doses caused more drastic and sustained reductions in blood glucose, which were reflected in the integrated areas above the curves. Error bars display s.e.m. (n=5). #p<0.05 by one-way ANOVA.

FIG. 15. Despite high bioactivity, intestinally administered insulin did not accumulate in peripheral blood. While subcutaneous injection caused a spike in blood insulin concentration followed by a rapid decline, intestinal administration after silica nanoparticles sustained modest elevations in serum levels, likely due to first-pass uptake by the liver. Error bars display s.e.m. (n=5).

FIG. 16. Silica nanoparticles enabled true oral insulin delivery in mice. Orally administered insulin capsules induced pronounced and sustained hypoglycemia at doses as low as 10 U/kg when co-administered with silica nanoparticles. Oral insulin without particles produced no effect compared to the inactive control protein BSA. Particle treatments resulted in multiple-fold increases in the area above the blood glucose curve calculated to 10 hours. Error bars display s.e.m. (n=5). #p<0.05 by one-way ANOVA.

FIG. 17. While subcutaneous injection caused a spike in blood exenatide concentration followed by a rapid decline, orally administered exenatide capsules with nanoparticle treatment sustained modest elevations in serum levels for several hours. Oral exenatide without particles did not accumulate in any significant levels in the blood stream. Error bars display s.e.m. (n=5).

FIG. 18. Silica nanoparticles increased permeability by binding cell surface integrins and inducing tight junction rearrangement. An integrin- and myosin light chain (MLC)-dependent cell signaling pathway has been previously linked to intestinal permeability. Here, nanoparticles of sufficiently small size and negative charge bind to intestinal epithelial integrins, opening tight junctions to allow absorption of protein drugs.

FIG. 19. Inhibiting the integrin/MLC pathway inhibited the absorption enhancing effect of silica nanoparticles. Blocking 2 out of the 24 known integrins reduced 50 nm silica efficacy by approximately 25%, while inhibition of pMLC activation completely prevented any particle-induced changes in monolayer permeability. Error bars display s.e.m. (n=3), *p<0.05 with respect to control wells.

FIG. 20. Silica nanoparticles did not infiltrate epithelial cells in vitro intestinal barriers. A side view confocal image of a nanoparticle-treated monolayer confirmed that nanoparticles localized on top of the cells but did not permeate into or through the tight junctions. Scale bar displays 10 μm. A sketch is included to clarify the different signals in the confocal image.

FIG. 21. Silica nanoparticles did not cross in vitro intestinal epithelia. When placed on top of Caco-2 monolayers, FITC-labeled silica nanoparticles (50 nm) did not cross the epithelial models. Particles accumulated in the basal chamber when no cells were present.

FIG. 22. Particles did not increase absorption of 40,000 kDa dextran (FITC-DX40). This indicates that the absorption enhancing effect of the silica nanoparticles is limited to smaller proteins and peptides, circumventing the risk of bacterial infiltration through the epithelium. Error bars display s.e.m. (n=6).

FIG. 23. Particle-induced permeability of mouse intestines was reversible. FITC-DX4 was not absorbed into the bloodstream when administered 24 hours after nanoparticle treatment. Error bars display s.e.m. (n=6). *p<0.05 by one-tailed t-test, with respect to PBS control mice.

FIG. 24. Histological analysis indicated no intestinal tissue damage due to treatment with silica nanoparticles. Neither untreated (left) nor 50 nm silica-treated (right) mice presented with intestinal infiltration of inflammatory cells or detectable changes in epithelial architecture.

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values.

As used herein stating that a layer is said to be disposed “over” a referenced layer, or “about a circumference of” a referenced layer, or “about at least a portion of the circumference of” a referenced layer, does not imply the layer is directly adjacent to the referenced layer, and may comprise one or more additional layers therebetween, and further does not imply that the layer completely covers the referenced layer, and may only cover, surround, contact, etc. only a portion of the referenced layer. That said, if a layer is said to be disposed “directly about” or “directly over” a referenced layer, it is meant the two layers contact each other, though an intermediary layer, such as an adhesive layer, or a blended layer that results from directly contacting the two layers during the process of formation of the device may be present between the two stated layers. Also, if a layer is said to “completely cover” a referenced layer, it is meant the second layer covers the entirety of the referenced layer. Stating that a layer is said to be disposed “over” another layer, or “about a circumference of” a referenced layer, or “about at least a portion of the circumference of” a referenced layer includes where the stated layers are directly contacting each other and/or that the layer completely covers the referenced layer.

As used herein, spatial or directional terms, such as “left”, “right”, “inner”, “outer”, “above”, “below”, “over”, “under”, and the like, relate to the invention as it is shown in the drawing figures are provided solely for ease of description and illustration, and do not imply directionality, unless specifically required for operation of the described aspect of the invention. It is to be understood that the invention can assume various alternative orientations and, accordingly, such terms are not to be considered as limiting.

As used herein, the terms “comprising,” “comprise” or “comprised,” and variations thereof, are meant to be open ended. The terms “a” and “an” are intended to refer to one or more.

As used herein, the “treatment” or “treating” of a condition means administration to a patient by any suitable dosage regimen, procedure and/or administration route of a composition, device or structure with the object of achieving a desirable clinical/medical end-point. An effective amount of an active ingredient, drug, etc. for treatment of a condition, is thus an amount of that active ingredient, as delivered, effective to treat a patient having that condition. For example, in the treatment of diabetes, e.g., type 1 diabetes or type 2 diabetes (type 1 diabetes mellitus or type 2 diabetes mellitus), insulin resistance, or pancreatic insufficiency, an effective amount of an active ingredient or drug is an amount that maintains appropriate regulation of glucose metabolism or glucose levels in that patient, with an exemplary end point being achievement or maintenance of blood glucose levels in a patient within a safe or appropriate range.

As used herein, the term “patient” or “subject” refers to members of the animal kingdom including but not limited to human beings and “mammal” refers to all mammals, including, but not limited to human beings.

A composition is “biocompatible” in that the composition and, where applicable, degradation products thereof, are substantially non-toxic to cells or organisms within acceptable tolerances, including substantially non-carcinogenic and substantially non-immunogenic, and are cleared or otherwise degraded in a biological system, such as an organism (patient) without substantial toxic effect. Non-limiting examples of degradation mechanisms within a biological system include chemical reactions, hydrolysis reactions, and enzymatic cleavage.

As used herein, the term “polymer composition” is a composition comprising one or more polymers. As a class, “polymers” includes, without limitation, homopolymers, heteropolymers, co-polymers, block polymers, block co-polymers and can be both natural and/or synthetic. Homopolymers contain one type of building block, or monomer, whereas copolymers contain more than one type of monomer. The term “(co)polymer” and like terms refer to either homopolymers or copolymers.

A polymer “comprises” or is “derived from” a stated monomer if that monomer is incorporated into the polymer. Thus, the incorporated monomer (monomer residue) that the polymer comprises is not the same as the monomer prior to incorporation into a polymer, in that at the very least, certain groups are missing and/or modified when incorporated into the polymer backbone. A polymer is said to comprise a specific type of linkage if that linkage is present in the polymer.

As used herein, a “peptide” is a chain of amino acids linked by an amide or peptide bond, and includes as a class oligopeptides and polypeptides. Oligopeptides are short peptides, typically referred to as having ten or less amino acids (amino acid residues). A “protein” comprises one or more peptide (polypeptide) chains, typically of 40 or more amino acids. Of note, depending on the nature of the composition comprising an amino acid chain, the terms “peptide”, “polypeptide”, and “protein”, may be interchangeable.

In one aspect, provided herein is a device, dosage form, or composition comprising negatively-charged nanoparticles having an average size, e.g., Z average determined by dynamic light scattering, of less than 1 μm, 500 nm, less than 200 nm, or less than 100 nm, e.g., 50 nm or 20 nm, an active ingredient of less than 40 kDa, 25 kDa, or 10 kDa, or a hydrodynamic radius of less than 10 nm, 4 nm, or 2 nm, and a pharmaceutically acceptable excipient. Non-limiting examples of the device, dosage form, or composition are provided below. Also provided herein is a trans-epithelial drug delivery method, comprising: contacting an epithelial membrane (epithelial tissue or epithelium) with a negatively-charged nanoparticle having an average size, e.g., Z average determined by dynamic light scattering, of less than 1 μm, 500 nm, less than 200 nm, or less than 100 nm, e.g., 50 nm or 20 nm; and then contacting the epithelial membrane with an active ingredient of less than 40 kDa, 25 kDa, or 10 kDa, or a hydrodynamic radius of less than 10 nm, 4 nm, or 2 nm. Also provided is a method of treating diabetes or insulin resistance, or inducing weight loss, in a patient, comprising administering an amount of insulin or a glucagon-like peptide-1 receptor agonist, such as exenatide, liraglutide; lixisenatide; albiglutide; dulaglutide; semaglutide; or taspoglutide, effective to treat diabetes or to induce weight loss. As is broadly-known, type 2 diabetes is characterized by insulin resistance, hyperglycemia, and the inability of the patient to produce sufficient insulin, and is often responsive to treatment with insulin or a glucagon-like peptide-1 receptor agonist, among other treatments. Insulin resistance is the failure of cells to properly respond to insulin—preventing glucose use, resulting in hyperglycemia. Insulin resistance may be found in pre-diabetic patients and patients suffering from metabolic syndrome. Type 1 diabetes results from the inability of a patient to produce sufficient amounts of insulin, and is treated by administration of insulin to the patient, traditionally by injection or insulin pump.

The devices, dosage forms, compositions, and methods described herein are useful for delivery of an active ingredient or drug to any epithelial membrane, including mucosal membranes. One or more active ingredients or drugs may be administered in a single device or dosage form, to achieve appropriate treatment of a condition, such as diabetes, e.g., type 1 or type 2 diabetes. For example, and without limitation, a glucagon-like peptide-1 receptor agonist, such as exenatide, and metformin may be included in the same dosage form and co-administered in amounts effective to treat diabetes. Other small molecules may be incorporated into the dosage forms for treatment of diabeties, including: gliclazide; pioglitazone; repaglinide; acarbose; or a gliptin (a dipeptidyl peptidase-4 (DPP-4) inhibitor, such as: sitagliptin; vidagliptin; saxagliptin; linagliptin; alogliptin; dutogliptin; or gemiglaptin).

Epithelial tissue, as referred to herein, includes all forms of epithelium, irrespective of derivation from ectoderm, endoderm, or mesoderm, and includes squamous epithelium, cuboidal epithelium, and columnar epithelium, including pseudostratified columnar epithelium. In one aspect, epithelium includes endothelium, which are squamous cells including blood vessel and lymphatic endothelium. Certain epithelial tissue may be ciliated or smooth. Broadly-known examples of epithelium include: simple squamous epithelium, including vascular and lymphatic endothelium; simple cuboidal epithelium; simple columnar epithelium; pseudostratified columnar epithelium; stratified squamous epithelium; stratified cuboidal epithelium; stratified columnar epithelium; and transitional epithelium. Epithelial tissue typically forms a membrane, referred to herein, as an “epithelial membrane”. The devices, dosage forms, drug products, and methods described herein facilitate passage of active ingredients (e.g., drugs, chemical entities, or biologicals) through epithelial tissue, e.g., epithelial membranes, and therefore facilitate trans-epithelial delivery of those active ingredients, thereby increasing bioavailability and therefore therapeutic efficacy of certain active ingredients previously not deliverable via trans-epithelial, e.g., intestinal, delivery of those active ingredients.

In aspects, the nanoparticles have an average diameter of less than 200 nm, or less than 100 nm, and an average diameter of greater than 10 nm or 20 nm. In one example, the nanoparticles have an average diameter of between 20 nm and 100 nm, or between 20 nm and 50 nm. As above, the diameter of the particles can be measured, and represented statistically, by any useful method.

Examples of useful negatively-charged nanoparticles include, without limitation: silica nanoparticles; metal nanoparticles, such as silver, gold, or platinum nanoparticles; metal oxide nanoparticles, such as cerium oxide, iron oxide, or titanium dioxide nanoparticles; nanoparticles consisting of any core that is surface-functionalized with negative moieties, such as carboxylic acid, glutathione, or dihydrolipoic acid; or polymer or polymer-coated nanoparticles. Polymers useful in polymeric or polymer-coated nanoparticles include, for example and without limitation, polystyrene, polylactic acid, polyglycolic acid, and polyesters. Importantly, the nanoparticles should be non-toxic to a patient.

The nanoparticles may be porous, for example, due to the size of the particles, mesoporous—having pore diameters in the range of 2 nm to 50 nm. Porous particles allow permeation or infiltration by active ingredients, such as small molecules or biological molecules, including polypeptides and proteins. During delivery of the porous nanoparticles containing the active ingredient, the active ingredient can elute from the nanoparticle, and depending on the particular combination of nanoparticle and active ingredient, the release profile of the active ingredient can be tailored, ranging from an immediate (bolus) release profile, to an extended release profile.

Silica refers to silicon dioxide. Silica can be formed into nanoparticles (particles having an average particle size of less than 1 micron (μm)). For purposes herein, a stated particle size is the Z-average diameter as determined by dynamic light scattering (DLS). This method and standards are broadly-known. Other methods may be used to determine particle size, and for certain types of nanoparticles may be more appropriate, and as such, the size limits presented may be applied to any useful method of size determination (see, e.g., Kato, H. et al., Determination of size distribution of silica nanoparticles: A comparison of scanning electron microscopy, dynamic light scattering, and flow field-flow fractionation with multi-angle light scattering methods. Mater. Express, 4(2):144-152 (2014)), including scanning electron microscopy (SEM), transmission electron microscopy (TEM), static light scattering, and other statistical representations of DLS data (e.g., intensity average).

Silica and other polymeric nanoparticles are a common additive in foods and other consumer products, and are thus known to be well tolerated by the human gastrointestinal tract. However, the step of co-delivering unbound nanoparticles to improve the bioavailability of oral macromolecules has not previously been explored, and may assist in delivering therapeutics without causing nanoparticle accumulation within the body.

Silica nanoparticles are examples of nanoparticles useful in the devices, dosage forms, and methods described herein. In other aspects, and in a broader context, negatively-charged nanoparticles are useful in the methods, compositions, and devices/dosage forms described herein. In one example, the nanoparticles are negatively charged in a range between pH 6 to pH 8, for example, at pH 7 or pH 7.4. The charge of a nanoparticle may be expressed in reference to its zeta potential (ζ-potential), which is typically measured in terms of millivolts (mV) and can be measured using any appropriate method. In aspects, the ζ-potential of the particles ranges from less than 0 mV to −80 mV, from −20 mV to −80 mV, or from −30 mV to −50 mV, such as −40 mV.

An “active ingredient” is any component that provides pharmacological activity or other direct effect in the diagnosis, cure, mitigation, treatment, or prevention of disease, or to affect the structure or any function of the body of man or animals. A drug is: a substance recognized by an official pharmacopoeia or formulary; a substance intended for use in the diagnosis, cure, mitigation, treatment, or prevention of disease; a substance (other than food) intended to affect the structure or any function of the body; a substance intended for use as a component of a medicine but not a device or a component, part or accessory of a device; or a biological product. A dosage form is the physical form in which a drug is produced and dispensed, such as a tablet, a capsule, or an injectable. A drug product is a finished dosage form that contains a drug substance, generally, but not necessarily in association with other active or inactive ingredients. A unit dosage form is a dosage form providing a single dose of an active agent. Oral dosage forms, such as tablets, capsules, suppositories, and the like, typically are provided as unit dosage forms. It is to be understood that the nanoparticles and active ingredients described herein can be provided in one single, unit dosage forms or separate (unit) dosage forms that can be taken, e.g., orally or as a suppository. However, in practice, it may be preferable to co-localize the nanoparticles and active ingredient at a single site on an epithelial membrane, and, as such, it may be preferred to incorporate both the nanoparticles and active ingredient in a single unit dosage form, such as a tablet, capsule, suppository, etc., e.g., with a mucoadhesive component, so that the nanoparticles and active ingredient are co-localized, thereby effectively increasing the local concentration of the nanoparticles and active ingredient. More than one active ingredient may be provided in a dosage form.

The size of the active ingredient delivered by the methods, compositions, and dosage forms described herein is relevant to the ability of that active ingredient to permeate epithelial tissue. In one aspect, the active ingredient (e.g., drug) is less than 40 kDa (kiloDaltons, either in molecular weight or, in the case of polydisperse compounds, number average molecular weight), e.g., ranging from 10 Da (Dalton) to less than 40 kDa, such as from 100 Da to 10 kDa, from 100 Da to 15 kDa, from 100 Da to 20 kDa, from 100 Da to 25 kDa, or from 100 Da to 30 kDa, e.g., from 5 kDa to 8 kDa, or from 3 kDa to 4 kDa.

In one aspect, the active ingredient is insulin, or an active analog or derivative thereof. In another aspect, the active ingredient is a glucagon-like peptide-1 receptor agonist (or incretin mimetic), such as exenatide, liraglutide; lixisenatide; albiglutide; dulaglutide; semaglutide; or taspoglutide. In one aspect, the active ingredient is exenatide (e.g., Exendin-4, HGEGTFTSDLSKQMEEEAVRLFIE-WLKNGGPSSGAPPPS-NH₂; (SEQ ID NO: 1)). In one aspect, the active ingredient is the peptide calcitonin, also known as thyrocalcitonin (human: CGNLSTCMLGTYT-QDFNKFHTFPQTAIGVGAP-NH₂ (SEQ ID NO: 2); salmon—CSNLSTCVLGKLSQE-LHKLQTYPRTNTGSGTP-NH₂ (SEQ ID NO: 3)).

In another aspect, the active ingredient is an antibody fragment that is less than 40 kDa, such as an Fv fragment or an scFv (single-chain variable fragment). In another aspect, the active ingredient is an aptamer.

Although compounds having low molecular weights (e.g., small molecules, examples of which are listed below) may be delivered by other dosage forms and routes, in instances, there may be benefits to using the method, composition, device, dosage form, or drug product described herein to deliver the low molecular weight molecules alone or in combination with other active ingredients, e.g., larger macromolecules, such as polypeptides, of less than 40 kDa. The active ingredient may be, without limitation, one or more of an antiseptic, an antibiotic, an analgesic, an anesthetic, a chemotherapeutic agent, a clotting agent, an anti-inflammatory agent, a metabolite, a cytokine, a chemoattractant, a hormone, a steroid, a protein, or a nucleic acid.

Active ingredients that may be incorporated, by themselves, or in combination with another active ingredient, such as a polypeptide, and/or a suitable excipient, into any composition, device, dosage form, or drug product described herein include, without limitation: anti-inflammatories, such as, without limitation, NSAIDs (non-steroidal anti-inflammatory drugs) such as salicylic acid, indomethacin, sodium indomethacin trihydrate, salicylamide, naproxen, colchicine, fenoprofen, sulindac, diflunisal, diclofenac, indoprofen sodium, salicylamide, anti-inflammatory cytokines, and anti-inflammatory proteins or steroidal anti-inflammatory agents; antibiotics and antivirals, such as, without limitation: acyclovir, afloxacin, ampicillin, amphotericin B, atovaquone, azithromycin, ciprofloxacin, clarithromycin, clindamycin, clofazimine, dapsone, diclazaril, doxycycline, erythromycin, ethambutol, fluconazole, fluoroquinolones, foscarnet, ganciclovir, gentamicin, iatroconazole, isoniazid, ketoconazole, levofloxacin, lincomycin, miconazole, neomycin, norfloxacin, ofloxacin, paromomycin, penicillin, pentamidine, polymixin B, pyrazinamide, pyrimethamine, rifabutin, rifampin, sparfloxacin, streptomycin, sulfadiazine, tetracycline, tobramycin, trifluorouridine, trimethoprim sulphate, Zn-pyrithione, ciprofloxacin, norfloxacin, afloxacin, levofloxacin, gentamicin, tobramycin, neomycin, erythromycin, trimethoprim sulphate, polymixin B and silver salts such as chloride, bromide, iodide and periodate; anticlotting factors such as heparin, Pebax, enoxaprin, aspirin, hirudin, bivalirudin, prasugrel, idraparinux, warfarin, clopidogrel, PPACK, GGACK, tissue plasminogen activator, urokinase, and streptokinase; growth factors; immunosuppressants; glucocorticoids such as hydrocortisone, betamethasone, dexamethasone, flumethasone, isoflupredone, methylpred-nisolone, prednisone, prednisolone, and triamcinolone acetonide; antiangiogenics, such as fluorouracil, paclitaxel, doxorubicin, cisplatin, methotrexate, cyclophosphamide, etoposide, pegaptanib, lucentis, tryptophanyl-tRNA synthetase, anecortave, CA4P, AdPEDF, VEGF-TRAP-EYE, Avastin, JSM6427, TG100801, ATG3, OT-551, endostatin, thalidomide, becacizumab, neovastat; antiproliferatives such as sirolimus, paclitaxel, perillyl alcohol, farnesyl transferase inhibitors, FPTIII, L744, antiproliferative factor, 5-FU, Daunomycin, Mitomycin, dexamethasone, azathioprine, chlorambucil, methotrexate, mofetil, vasoactive intestinal polypeptide, and PACAP; antibodies and fragments thereof; antigens for vaccinations, including virus capsid proteins and fragments thereof; drugs acting on immunophilins, such as cyclosporine, zotarolimus, everolimus, tacrolimus and sirolimus (rapamycin), interferons, or TNF binding proteins; taxanes, such as docetaxel; statins, such as atorvastatin, lovastatin, simvastatin, pravastatin, fluvastatin, and rosuvastatin; or nitric oxide donors or precursors, such as, without limitation, Angeli's Salt, L-Arginine, Free Base, Diethylamine NONOate, Diethylamine NONOate/AM, Glyco-SNAP-1, Glyco-SNAP-2, S-Nitroso-N-acetylpenicillamine, S-Nitrosoglutathione, NOC-5, NOC-7, NOC-9, NOC-12, NOC-18, NOR-1, NOR-3, SIN-1, Sodium Nitroprusside, Spermine NONOate, Streptozotocin; peptide hormones, such as amylin, angiotensin, calcitonin, endothelin, glucagon, glucagon-like-peptide-1, human chorionic gonadotropin, human placental lactogen, growth hormone, insulin, insulin-like growth factor, luteinizing hormone, oxytocin, prolactin, relaxin, secretin, thyroid-stimulating hormone.

Examples of active ingredients (e.g., drugs) that are presumed to be deliverable by the methods, compositions, devices, dosage forms, or drug products described herein, include: biologics; proteins; peptides; nucleic acids, including nucleic acid analogs such as DNA, RNA, peptide nucleic acid (PNA), short interfering RNA (siRNA), messenger RNA (mRNA), microRNA (miRNA), tRNA, phosphorothioate, locked nucleic acid, unlocked nucleic acid, 2′-O-methyl-substituted RNA, morpholino nucleic acid, threose nucleic acid, glycol nucleic acid backbone, or modified RNA bases, including pseudouridine, 1-methylpseudouridine, 5-methylcytidine, or 2-thiouridine, or any combination thereof, including aptamers, as are broadly-known. Nucleic acid analogs also include peptide nucleic acids, such as γ-peptide nucleic acids. As indicated above, the active ingredients have a molecular weight (for a defined compound, including defined proteins, peptides, nucleic acids, or aptamers, or a number average molecular weight Mn for a polydisperse compound or composition, such as a polymer or a polymer-modified protein, peptide, aptamer or small molecule) of less than 40 Da, 35 Da, 30 Da, 20 Da, 15 Da, or increments therebetween, and no effective minimal molecular weight, though greater than 1 Da, and more realistically, greater than 50 Da or 100 Da.

Specific active ingredients deliverable by the methods, compositions, devices, dosage forms, or drug products described herein include, without limitation: abaloparatide, adrenocorticotropic hormone, afamelanotide, albiglutide, ambamustine, atosiban, aviptadil, buserelin, carbetocin, carfilzomib, carperitide, cetrorelix, cholecystokinin, calcitonin (salmon or human), carperitide, corticotropin, cyclosporine, degarelix, desmopressin, dulaglutide, elcatonin, eledoisin, enalapril, enfuvirtide, etelcalcetide, exenatide, felypressin, ganirelix, glatiramer, glucagon, glucagon-like peptide 2, glucose-dependent insulinotropic peptide, gonadorelin, goserelin, heparin, histrelin, human growth hormone, icatibant, insulin, lanreotide, leuprorelin (leuprolide), linaclotide, liraglutide, lisinopril, lixisenatide, lucinactant, lutetium, lypressin, mifamurtide, nafarelin, nesiritide, octreotide, ornipressin, oxytocin, pasireotide, plecanatide, pramlintide, romiplostim, romurtide, somatostatin, taltirelin, teduglutide, teriparatide, terlipressin, tetracosactide, thymopentin, triptorelin, vasopression, virus capsid proteins and other antigens, voclosporin, or ziconotide.

A dosage form comprising the negatively-charged nanoparticles and an active ingredient, can be delivered in any useful fashion, including, without limitation, oral, sublingual, nasal, pulmonary, topical (including by rectal, vaginal, or urethral suppository), intravenous, or ophthalmic administration. In one aspect, the negatively-charged nanoparticles and active ingredient are delivered in a liquid or encapsulated, such as in a gelatin capsule or enteric-coated capsule, as are broadly-known. It may be preferable, in many instances, to provide a delayed-release product in which the negatively-charged nanoparticles and an active ingredient are released in the intestine, and therefore the coating is an enteric coating. Oral coatings and delivery systems, e.g., enteric coatings, that release their active ingredient within the intestine are known and can be adapted to the dosage forms and methods as described herein. Suitable topical delivery systems also are known. See, e.g., Remington: The Science and Practice of Pharmacy, 21st edition, Ed. Paul Beringer et al., Lippincott, Williams & Wilkins, Baltimore, MD Easton, Pa. (2005) (see, e.g., Chapters 43-47 for examples of ophthalmic, topical and oral dosage forms, formulations and formulation methods).

The nanoparticles and active ingredients may be compounded or otherwise manufactured into a suitable composition for use, such as a pharmaceutical dosage form or drug product. Compositions may comprise a pharmaceutically acceptable carrier, or excipient. An excipient is an inactive substance used as a carrier for the active ingredients of a medication. Although “inactive,” excipients may facilitate and aid in increasing the delivery or bioavailability of an active ingredient in a drug product. Non-limiting examples of useful excipients include: antiadherents, binders, rheology modifiers, coatings, disintegrants, emulsifiers, oils, buffers, salts, acids, bases, fillers, diluents, solvents, flavors, colorants, glidants, lubricants, preservatives, antioxidants, sorbents, vitamins, sweeteners, etc., as are available in the pharmaceutical/compounding arts. Useful dosage forms include: liquids, oral tablets or capsules, topical ointments or creams, suppositories, and transdermal devices (e.g., patches).

In one aspect, the dosage form is a transdermal or transepithelial device, or “patch”. The general structure of a transdermal patch is broadly known in the pharmaceutical arts. A typical patch includes, without limitation: a delivery reservoir for containing and delivering a drug product to a subject, an occlusive backing to which the reservoir is attached on a proximal side (toward the intended subject's skin or epithelial layer) of the backing and extending beyond, typically completely surrounding the reservoir, and an adhesive on the proximal side of the backing, surrounding the reservoir, typically completely, for adhering the patch to the skin of a patient. For a transdermal system, the reservoir often comprises a matrix formed from a non-woven (e.g., a gauze) or a hydrogel, such as a polyvinylpyrrolidone (PVP) or polyvinyl acetate (PVA), as are broadly known. The reservoir comprises the active ingredient absorbed into or adsorbed onto the reservoir matrix, the negatively-charged nanoparticles, and optionally, a chemical permeation enhancer.

In one aspect, the dosage form is a mucoadhesive patch for delivery to the mucosa, and including the nanoparticles and active ingredient embedded separately or together in a mucoadhesive composition. In use, the mucoadhesive patch is delivered, for example and without limitation, orally, ophthalmically, or by suppository. The patch adheres to mucosa, and slowly dissolves, releasing the nanoparticles and active ingredient—thereby delivering those constituents locally in the intestine, or to other mucosa. The mucoadhesive patch may be encapsulated for oral delivery in an enteric coating, as are broadly-known, that dissolves over time to release the patch in a patient's intestine.

Drug products, such as patches or mucoadhesive patches, comprising the nanoparticles and an active ingredient, optionally include a chemical permeation enhancer. A chemical permeation enhancer (CPE) is a chemical that aids transport across the epithelium by altering the structure of the cellular membrane (transcellular route) and/or the tight junctions between cells (paracellular route) of the epithelium. CPEs possess a broad range of chemical structures (See, e.g., Table 1 of United States Patent Application Publication No 2015/0238435 A1, incorporated herein by reference for its technical disclosure). Many CPEs are small molecules. Chemical categories of such CPEs include: anionic surfactants (AS), cationic surfactants (CS), zwitterionic surfactants (ZS), nonionic surfactants (NS), bile salts (BS), fatty acids (FA), fatty esters (FE), fatty amines (FM), sodium salts of fatty acids (SS), nitrogen-containing rings (NR), and others (OT). The choice of chemical permeation enhancer typically depends on empirical studies. Non-limiting examples of permeation enhancers include: phenylpiperazine, methylpiperazine, sodium laureth sulfate, menthone, palmityldimethyl ammonio propane sulfonate, and N-lauryl sarcosinate.

As described above, and in United States Patent Application Publication No. 2015/0238435 A1, mucoadhesive dosage forms may be used for delivery the nanoparticles and active ingredient to a patient's intestines.

Mucoadhesives, e.g., mucoadhesive polymers, include, without limitation, polyanhydrides, polysaccharides, polymers and copolymers of acrylic acid, methacrylic acid, and their lower alkyl esters, for example polyacrylic acid, poly(methyl methacrylates), poly(ethyl methacrylates), poly(butylmethacrylate), poly(vinyl pyrrolidone), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate), Carbopol®, pectin, chitosan, carboxy methyl cellulose (CMC), hydroxy methyl cellulose (HMC), hydroxy ethyl cellulose (HEC), sodium carboxy methyl cellulose (SCMC), hydroxypropyl methylcellulose (HPMC). The mucoadhesive comprises any suitable, biocompatible mucoadhesive material. In one aspect, the mucoadhesive contains one or more of Carbopol® polymer, pectin, and a modified cellulose, such as Carbopol® 934 (BF Goodrich Co., Cleveland, Ohio), pectin (Sigma Chemicals, St. Louis, Mo.), and sodium carboxylmethylcellulose (SCMC, Aldrich, Milwaukee, Wis.). Carbopol® is a family of crosslinked acrylic acid-based polymers (poly(acrylic acid)) and copolymers, for example, acrylic acid, and optionally, a C₁₀-C₃₀ alkyl acrylate, crosslinked with allyl pentaerythritol. The mucoadhesive may further comprise a targeting moiety to facilitate targeting of the agent to a specific site in vivo. The targeting moiety may be any moiety that is conventionally used to target an agent to a given in vivo site such as an antibody, a receptor, a ligand, a peptidomimetic agent, an aptamer, a polysaccharide, a drug, or a product of phage display. In use, mucoadhesives eventually dissolve, disperse, or otherwise disengage from mucosa, releasing all or at least part of their contents, e.g., nanoparticles and, optionally, therapeutic ingredients, over time prior to their complete dissolution, dispersion, or disengagement.

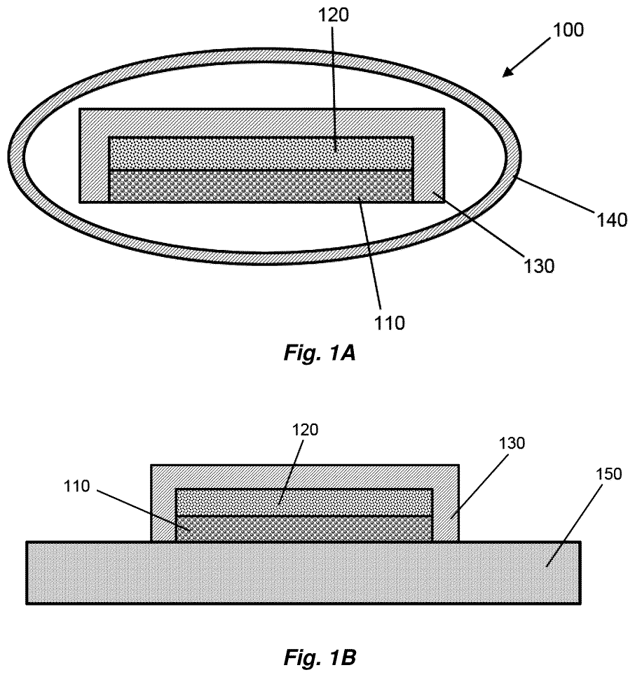

In aspects, shown in FIGS. 1A-3B, various mucoadhesive patches can be used to deliver therapeutic agents to mucosa, such oral, intestinal, rectal, or vaginal mucosa. FIGS. 1-3 are schematic in nature and elements thereof are not to scale, but are presented for ease of illustration. With respect to FIGS. 1A and 1B, dosage form 100, comprises a first mucoadhesive layer 110 comprising negatively-charged nanoparticles embedded within a mucoadhesive composition, a second layer 120 comprising a therapeutic agent embedded in a mucoadhesive composition, an impermeable backing 130 facilitates unidirectional diffusion of drug as well as preventing enzymatic degradation from the lumenal side of the patch, and a coating 140, such as an enteric coating or a gelatin coating. The term “embedded” does not infer or imply any method of “embedding” the nanoparticles in the mucoadhesive, but only refers to the nanoparticles being contained or distributed within the mucoadhesive, which can be achieved by any method, such as mixing. Layers 110 and 120, combined with backing 130 form a mucoadhesive patch, and the coating 140 typically completely covers or surrounds the mucoadhesive patch. As depicted in FIG. 1B, once the coating 140 dissolves, the first mucoadhesive layer 110 adheres to the intestinal surface 150 (depicted in part), and, first, the nanoparticles are released, and then the therapeutic ingredient in the second layer 220 is released. Once the mucoadhesive in the drug product is dissolved, the backing is released and passes through the intestine. Layer 120 may be a mucoadhesive in which the therapeutic ingredient is mixed, absorbed, adsorbed, or otherwise dispersed, or in other aspects, is a non-woven or other matrix suitable for reservoirs in transdermal devices, such as a PVA or PVP matrix. Additional layers, such as a microporous membrane, as is common to transdermal devices, may be included between layers 110 and 120. Dosage form 100 comprises non-biodegradable or non-dissolving elements, such as the backing 130, and in instances layer 120, and is more suitable for use where the dosage form is voidable, as in the gastrointestinal tract or urethra where the remaining elements of the device can be voided with feces or urine, respectively, as opposed to use in mucosa in locations that will not necessarily void the backing, etc., in a timely manner, such as with the vaginal mucosa or nasal mucosa.

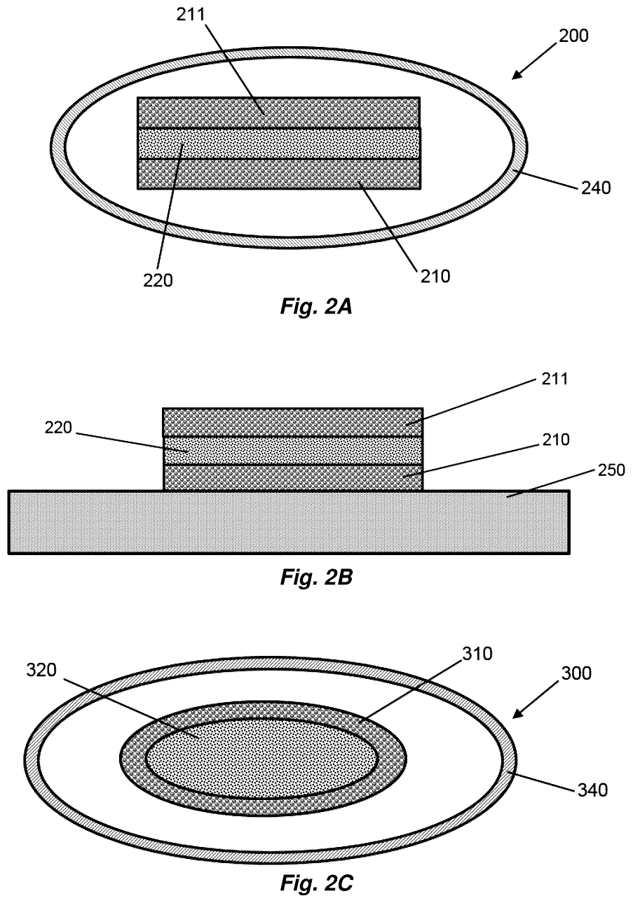

In a second aspect, depicted in FIGS. 2A and 2B, dosage form 200, comprises a first and a second mucoadhesive layer 210 and 211, respectively, comprising negatively-charged nanoparticles embedded within a mucoadhesive composition, a second layer 220 comprising a therapeutic agent is located between the first mucoadhesive layer 210 and the second mucoadhesive layer 211. Dosage form 200 also includes a coating 240, such as an enteric coating or a gelatin coating. Layers 210, 211, and 220 form a mucoadhesive patch, and the coating 240 typically completely covers or surrounds the mucoadhesive patch. As depicted in FIG. 2B, once the coating 240 dissolves, either the first mucoadhesive layer 210 or the second mucoadhesive layer 211 adheres to the intestinal surface 250 (depicted in part), and, first, the nanoparticles are released, and then the therapeutic ingredient in the second layer 220 is released. A benefit of this structure is that either side of the mucoadhesive patch can adhere to the mucosa. A variation of the dosage form 200 shown in FIG. 2A is depicted in FIG. 2C, where the dosage form 300 comprises a mucoadhesive layer 310 comprising negatively-charged nanoparticles embedded within a mucoadhesive composition, surrounding, optionally completely surrounding, a core 320 of a mucoadhesive composition comprising the therapeutic agent. A coating 340 also is depicted, such as an enteric coating or a gelatin coating. Layer 310 and core 220 form a mucoadhesive patch, and the coating 340 typically completely covers or surrounds the mucoadhesive patch.

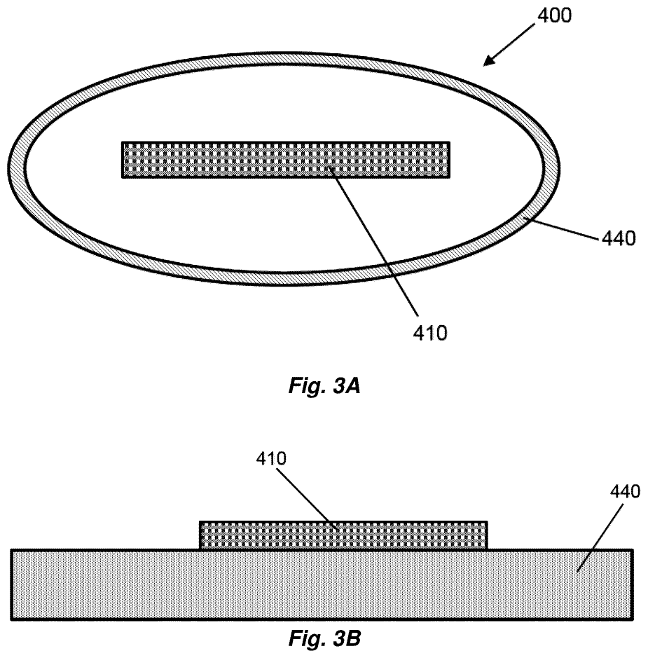

In another aspect, as depicted in FIGS. 3A and 3B, dosage form 400 comprises a mucoadhesive patch 420, comprising negatively-charged nanoparticles and a therapeutic agent contained within a mucoadhesive composition and surrounded by a coating 440, such as an enteric coating or a gelatin coating. The coating 440 typically completely covers or surrounds the mucoadhesive patch 420. As shown in FIG. 3B, the patch 410 adheres to intestinal mucosa 440 (shown in part), and dissolves, releasing the nanoparticles and the therapeutic agent.

With respect to FIGS. 1A, 2A, 2C, and 3A, the coating may be an enteric coating for an oral dosage form that releases the internal mucoadhesive patch in a patient's intestine, so that the patch adheres to intestinal mucosa. In other aspects, the coating is suitable for use in a suppository, for rectal, vaginal, or urethral delivery, with rapid dissolution of the coating and adhesion of the mucoadhesive patch to mucosa of the rectum, vagina, or urethra. The mucoadhesive patch may be rolled or folded within the coating to reduce its profile. In other aspects, the mucoadhesive patch is not coated, and can be used intranasally, orally (e.g., beneath the tongue or between the cheek and gum), or ophthalmically.

Work on the present invention has shown that silica and polystyrene nanoparticles at relevant concentrations (0.2 wt. %) are nontoxic to cells of the Caco-2 intestinal epithelial line. Trans-epithelial electrical resistance (TEER), calcein permeability, and dextran permeability assays have shown that the permeability of the Caco-2 monolayer increases when exposed to non-functionalized or surface carboxylated silica nanoparticles, both negative in charge. Further, oral delivery studies in mice have shown that pre-treatment with silica nanoparticles boosts the uptake of the model drug dextran and improves the uptake and efficacy of orally delivered insulin and exenatide, two commercially relevant protein drugs. In addition to being statistically significant and reproducible, silica nanoparticle-induced changes to barrier function and delivery efficacy were reversible and selective to particular sizes of delivery compounds.

### Example 1

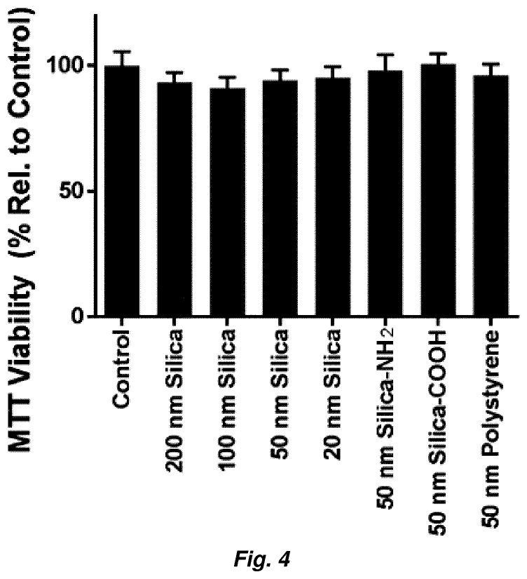

A non-limiting example of the present invention includes employing silica nanoparticles increase the permeability of both the Caco-2 monolayer intestinal model and of mouse intestines. Nanoparticles were synthesized by and purchased from two commercial suppliers, and their characteristics confirmed using a Malvern Zetasizer (Table 1). Each particle suspension was combined with cell culture media to produce a final particle concentration of 0.2% by weight, then screened for cytotoxicity to Caco-2 cells using the MTT viability assay (FIG. 4). None of the particle treatments tested showed any statistically significant reduction in cell viability.

| Core | | | | Z-average | | Zeta | | |
|---|---|---|---|---|---|---|---|---|
| Size | | Surface | | diameter | | potential | Particles per mass | Surface area |
| (nm) | Material | chemistry | Nomenclature | d · nm | PDI | (mV) | #/mg | cm\hat{ }2/mg |
| 200 | silica | n.f. | 200 nm SiO⁻ | 209 | 0.02 | −57.6 | 1.09E+11 | 137 |
| 100 | silica | n.f. | 100 nm SiO⁻ | 90 | 0.05 | −41.2 | 9.61E+11 | 280 |
| 50 | silica | n.f. | 50 nm SiO⁻ | 49 | 0.04 | −41.4 | 7.81E+12 | 554 |
| 20 | silica | n.f. | 20 nm SiO⁻ | 26 | 0.05 | −57.6 | 7.89E+13 | 1212 |
| 50 | silica | COOH | 50 nm SiO—COO⁻ | 46 | 0.15 | −27.3 | n/a | n/a |
| 50 | silver | PVP | 50 nm Ag³¹ | 54 | 0.14 | −21.4 | 1.47E+12 | 109 |
| 50 | gold | PVP | 50 nm Au³¹ | 61 | 0.09 | −16.3 | 7.64E+11 | 59 |
| 50 | polystyrene | n.f. | 50 nm PS | 53 | 0.06 | 0.2 | n/a | n/a |
| 50 | silica | NH₂ | 50 nm SiO—NH₃⁺ | 49 | 0.44 | 15.6 | 7.24E+12 | 547 |
| 50 | silica- | | 50 nm FITC-SiO⁻ | 56 | 0.20 | −27.2 | n/a | n/a |
| | FITC | | | | | | | |
| 20 | silica- | n.f. | 20 nm RhodB-SiO⁻ | 23 | 0.20 | −24.3 | n/a | n/a |
| | RhodB | | | | | | | |

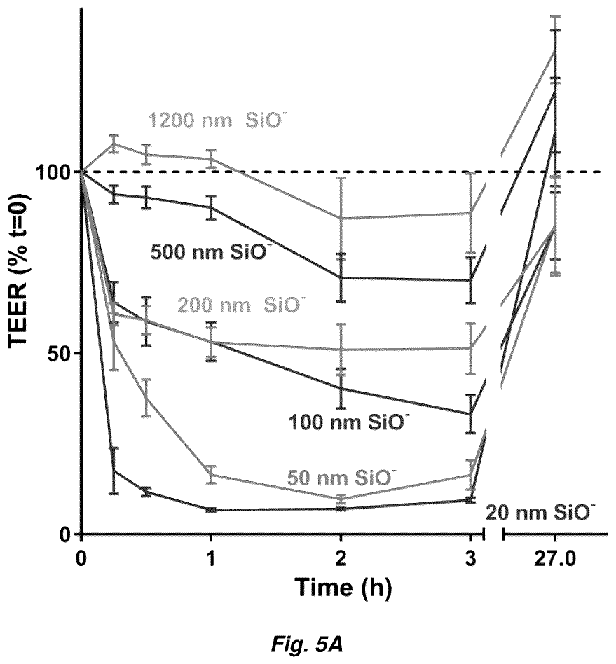

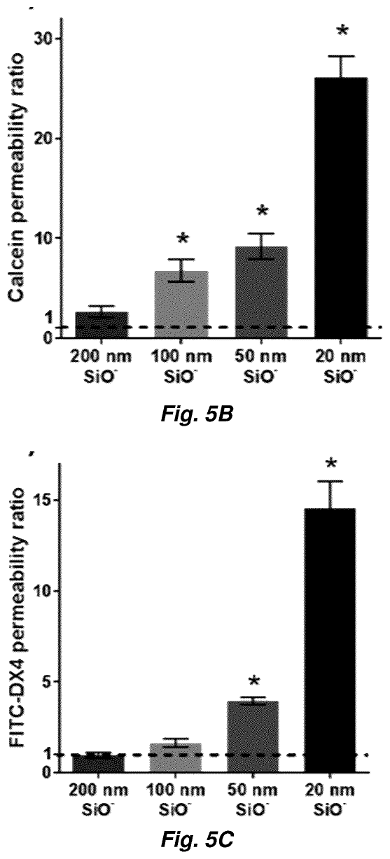

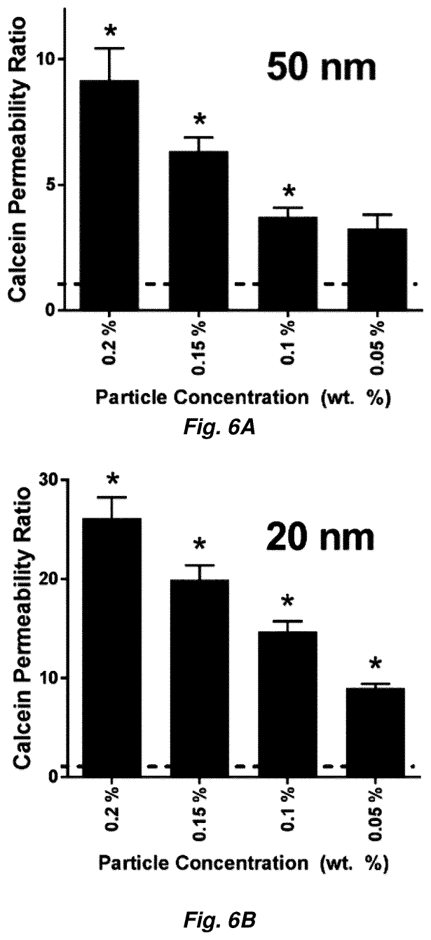

Multiple assays were performed to assess the permeability of the Caco-2 cell monolayer when exposed to the particle treatments over time. First, a TEER assay compared the effects of differently sized, non-functionalized (bare) silica nanoparticles. Among the four sizes of particles examined, there was a clear trend in which smaller particles were more effective at reducing TEER values, implying more permeable monolayers (FIG. 5A). This trend was confirmed by the increased passage of the permeation marker calcein (FIG. 5B) and the mock macromolecular drug 4 kDa, FITC-conjugated dextran (FITC-DX4) (FIG. 5C) through monolayers treated with smaller nanoparticles. Importantly, the monolayers also exhibited a dose-dependent response to both the 50 nm (FIG. 6A) and 20 nm (FIG. 6B) particle treatments, with higher concentrations of silica inducing a greater increase in permeability. This dose dependence confirmed that permeation enhancing abilities are a function of the silica nanoparticles themselves, not a particular assay condition or inactive component of the treatments.

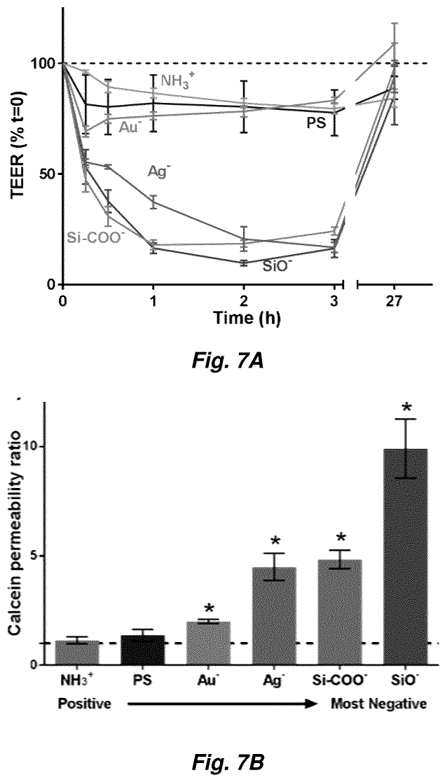

A second set of Caco-2 assays compared the effects of different particle surface chemistries and charges on permeabilization of intestinal cell monolayers. 50 nm particles of non-functionalized (“bare”) silica (negative), carboxylated silica (negative), polystyrene (neutral) and aminated silica (weak positive) were applied to the monolayer models, and only the negatively charged nanoparticles significantly reduced TEER values (FIG. 7A). This result was reflected in the significantly higher transport (when compared to controls) of calcein across only the monolayers treated with negatively charged nanoparticles (FIG. 7B).

In addition to permeability screening, Caco-2 monolayers were employed to visualize the cellular effects of nanoparticle treatments on the intestines. When compared with untreated controls (not shown), monolayers treated with 50 nm nanoparticles showed a rearrangement of the tight junction protein ZO-1 (not shown). Specifically, the treated monolayers developed clusters of cells between which the normally expressed ZO-1 borders disappeared. Since ZO-1 directly supports proteins of the tight junctions, which provide a strong barrier to macromolecular passage between the cells, we hypothesize that these areas of depleted protein expression act as improved diffusion pathways for our permeation enhancer and model drug. This particular type of localized ZO-1 disappearance has not been previously reported in literature.

In further detail, Caco-2 cells were imaged via confocal microscopy at 63× magnification for the tight junction protein ZO-1 at the apical surface, as well as actin and nucleic acids in the middle of the cells, approximately 2 μm below the apical surface. When compared with untreated monolayers, monolayers treated with 50 nm silica nanoparticles exhibited many communities of several cells inside which the tight junctions were not normally expressed. By overlaying the images of the tight junctions onto those of nuclei and actin in the same spot, it was seen that, while cell components did not map perfectly in the vertical projection, the 1:1 ratio of cells as visualized by tight junctions or nuclei/actin in the controls was not maintained in the nanoparticle treated cells, where several nuclei were bounded by one tight junctional boundary.

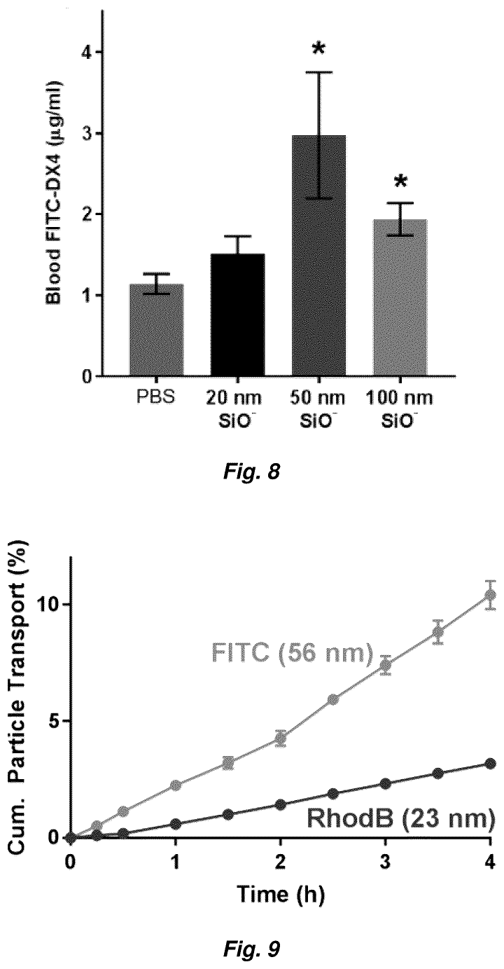

To confirm that permeation enhancement in Caco-2 correlates to improved macromolecular bioavailability in complex organisms, we orally dosed mice with silica nanoparticles, followed by (non-digestible) FITC-DX4. After three hours, serum fluorescence was measured to determine the blood FITC-DX4 concentration. As expected, the 50 and 100 nm particles increased FITC-DX4 absorption across the intestinal barrier (FIG. 8) when compared to a phosphate-buffered saline (PBS) gavage. However, despite being the most effective treatment in vitro, the 20 nm silica nanoparticles did not significantly increase FITC-DX4 uptake in mice.

It was hypothesized that the difference between in vitro and in vivo efficacy of 20 nm particles resulted from their interactions with the mucus layer that covers the epithelium in live animals but is not present in the Caco-2 model. To test this, some particles were tested for transport through an in vitro mucus model. A Type II mucus layer was placed on top of a permeable support, and internally fluorescent, non-surface-functionalized silica particles were tracked for their bulk movement through the barrier. Interestingly, the smaller particles (˜20 nm) were less able to diffuse through the mucus than the larger particles (˜50 nm) (FIG. 9). This difference likely causes a discrepancy in efficacy between the two particle sizes in living systems, and we hypothesized that it was due to differential mucus-binding behavior between the two sizes of particles.

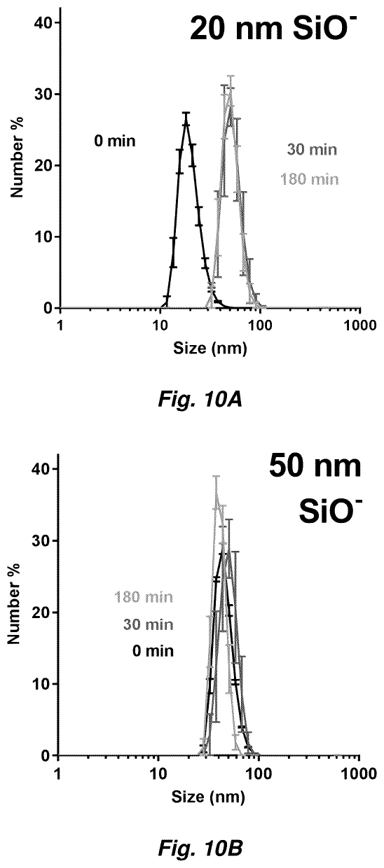

Mucus-binding particles are known to grow in apparent size when incubated with a dilute mucin suspension. Accordingly, 20, 50, and 100 nm silica particles were each mixed with a 1% (w/v) solution of type II mucin proteins and tracked their size over time via DLS. As shown in FIGS. 10A-10C, the 20 nm particles grew to approximately three times their original size within thirty minutes, indicating that these particles readily bind to intestinal mucus. By contrast, the non-binding 50 nm and 100 nm silica particles did not increase in apparent size.

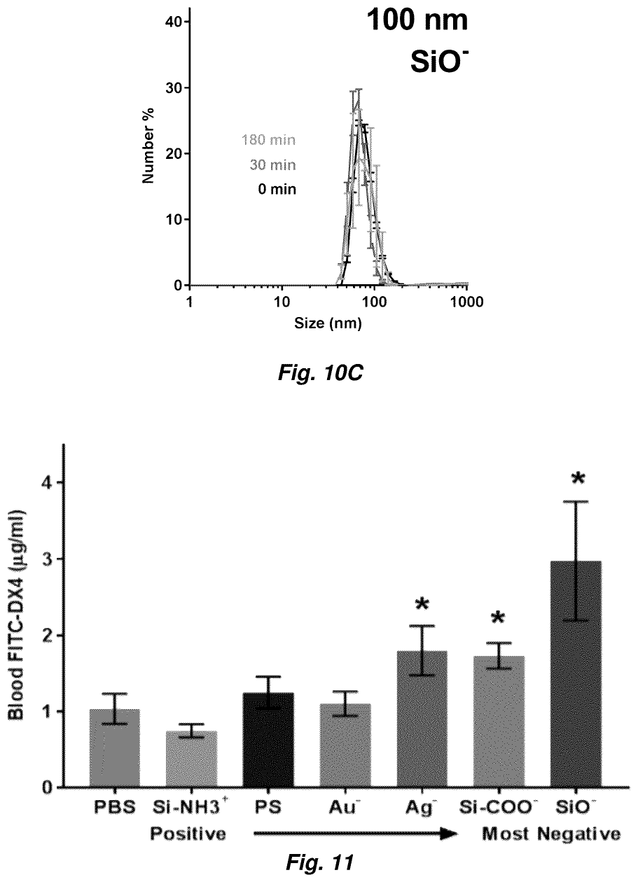

The link between efficacy and negative surface charge in mouse intestines was confirmed using FITC-DX4 and a collection of 50 nm particles with varied surface chemistry. As predicted, there was a trend between greater intestinal absorption of FITC-DX4 and stronger negative charge (FIG. 11), with neutral and positively charged particles causing no significant change in uptake. Further, the silica nanoparticles remained the most effective particle species at promoting oral macromolecular absorption.

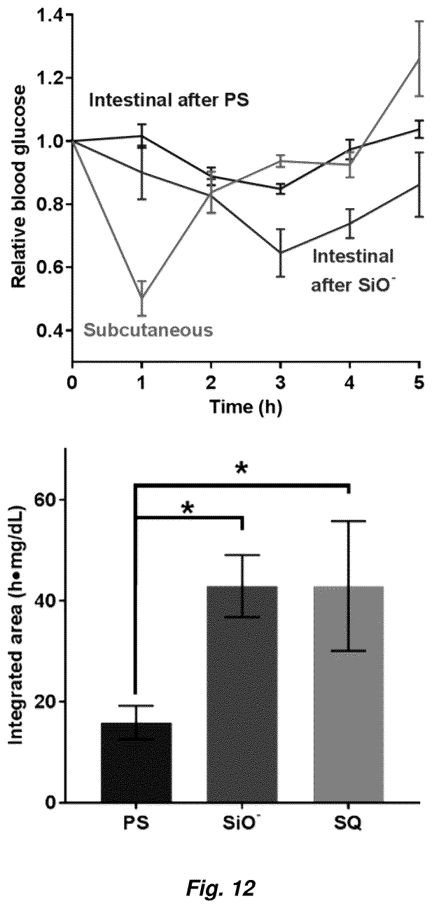

It was established that a protein drug could maintain its activity through silica-enabled intestinal translocation. Mice were orally gavaged with nanoparticles, then surgically administered a 1 U/kg dose of insulin directly into the small intestine, circumventing digestion in the stomach. Blood glucose levels were monitored in each mouse, and normalized to the mouse's blood sugar before the procedure. Mice that received insulin after 50 nm silica nanoparticle treatment showed a significant reduction in blood glucose when compared to mice that received the drug after polystyrene nanoparticles (FIG. 12). Further, the silica nanoparticle and insulin combination sustained hypoglycemia several hours longer than the same 1 U/kg dose of subcutaneous insulin. To compare the total insulin bioactivity between these administration methods, the area above each mouse's blood glucose curve that fell below the starting value were integrated. The areas above the curve (AACs) more clearly demonstrate that pharmacodynamic activity is approximately equal for subcutaneous and silica particle-assisted, intestinal insulin.

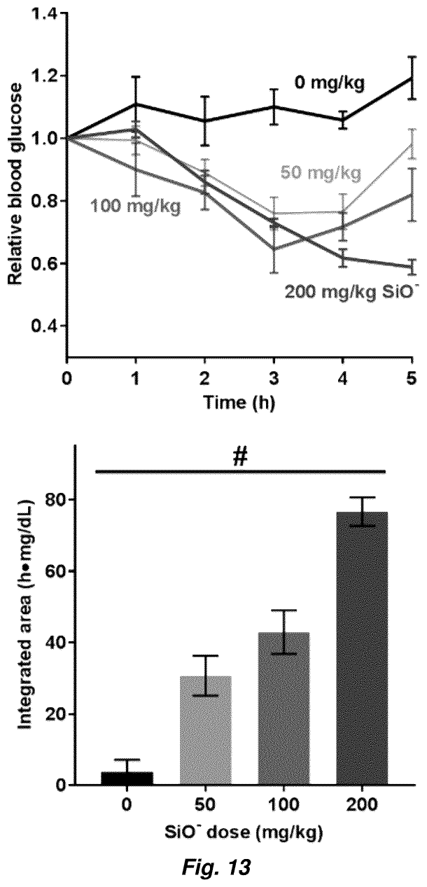

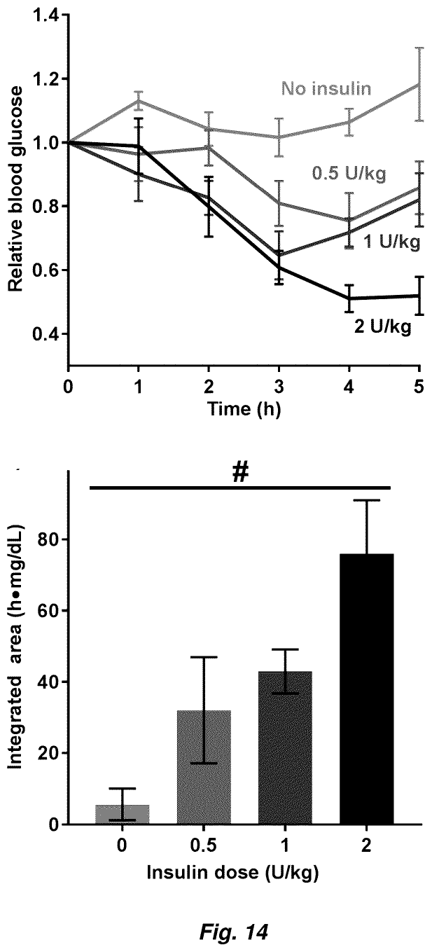

To ascertain that the observed hypoglycemia was a result of silica nanoparticles improving absorption of intestinal insulin, not a procedural artifact, dose responsiveness was examined. Increasing particle doses with constant insulin dose (1 U/kg for all groups) led to both greater magnitude and longer-sustained reductions in blood glucose levels (FIG. 13), reflecting strongly in the AACs. Likewise, with a constant particle administration of 100 mg/kg, increasing insulin dose correlated with increased magnitude and duration of hypoglycemia (FIG. 14). Together, these data confirm that improved insulin bioactivity is a result of nanoparticle-aided absorption in the intestines, and not of experimental conditions or animal reactions to handling.

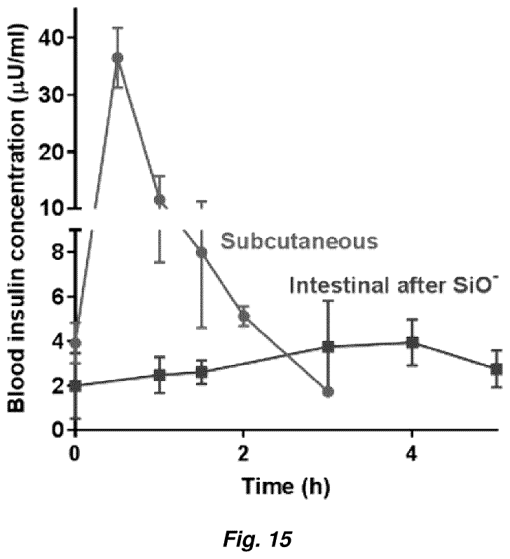

Having demonstrated strong, nanoparticle-induced improvement in pharmacodynamics, the pharmacokinetics of intestinally administered insulin were examined. Mice that received subcutaneous injections showed large spikes in blood insulin concentration within 30 minutes that returned to normal levels shortly after two hours (FIG. 15). By contrast, mice that received intestinal insulin along with nanoparticles demonstrated slight elevations in blood insulin that persisted for at least four hours. This apparent discrepancy between insulin activity and systemic insulin concentration is common among oral insulin uptake, and is likely due to first-pass liver uptake of insulin absorbed by the intestines.

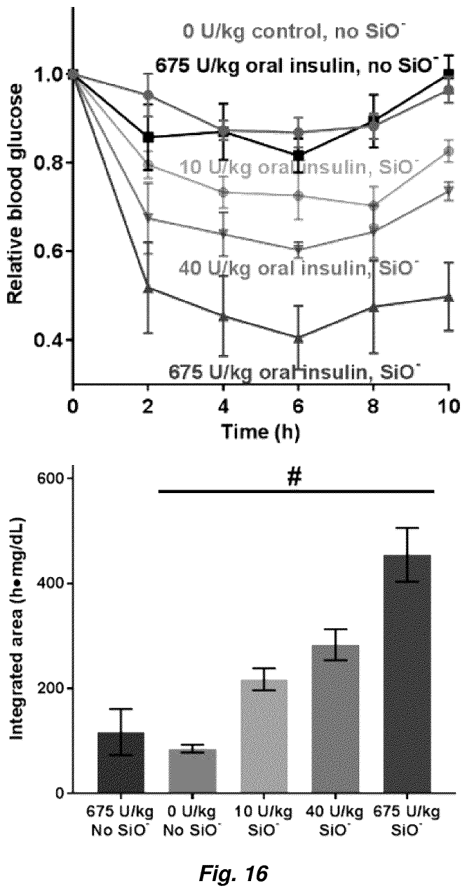

Next, insulin was delivered using silica nanoparticles in true oral fashion. Powdered insulin was combined with the inert protein bovine serum albumin (BSA) and the protease inhibitor aprotinin, which improves insulin survival in digestive fluids. This mixture was loaded into mouse (Size M) gel capsules at three different doses: 675 U/kg to observe maximal changes in blood sugar despite dissolution and localization challenges, 40 U/kg to more closely compare with other oral insulin systems, and 10 U/kg to probe how small of an oral insulin dose can be administered while still seeing therapeutic effect. The capsules were coated with Eudragit L100-55 to provide pH-responsive release in the small intestine. When delivered orally to mice with 50 nm silica nanoparticles, these insulin capsules evoked intense, sustained hypoglycemia that lasted at least ten hours past administration (FIG. 16). In contrast, insulin capsules given to mice without nanoparticle treatments caused no difference in blood glucose levels or corresponding AACs when compared to control capsules containing only BSA and aprotinin. Importantly, the 10 U/kg capsules demonstrated approximately 35% bioactivity on a per dose basis with respect to the subcutaneous injection. This is a major improvement compared with the 2-10% relative bioactivity seen with other promising oral delivery systems.

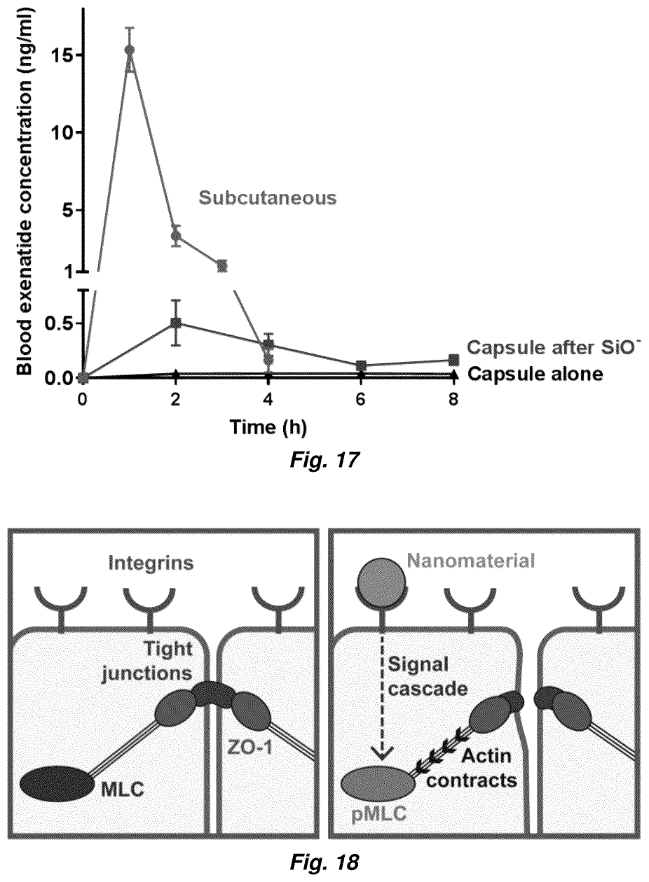

To demonstrate this invention's utility among other peptide drugs, the pharmacokinetics of orally administered exenatide was examined. Exenatide capsules were prepared in the same fashion as the oral insulin capsules, giving a final exenatide dose of 1 mg/kg. After capsule administration, blood was drawn every two hours, for a total of eight hours, and assayed the serum for exenatide concentration using ELISA. As before, mice that received subcutaneous injections showed large spikes in serum drug concentration within 30 minutes that returned to normal levels shortly within four hours (FIG. 17). By contrast, mice that received oral exenatide along with nanoparticles demonstrated a significant increase in blood exenatide levels (over oral exenatide with no particles) for at least eight hours. Once again, exenatide levels in the peripheral blood may appear low due to first-pass liver processing of all material absorbed by the intestines. However, the particle treatments still resulted in an orders-of-magnitude higher bioavailability with respect to the oral exenatide alone.

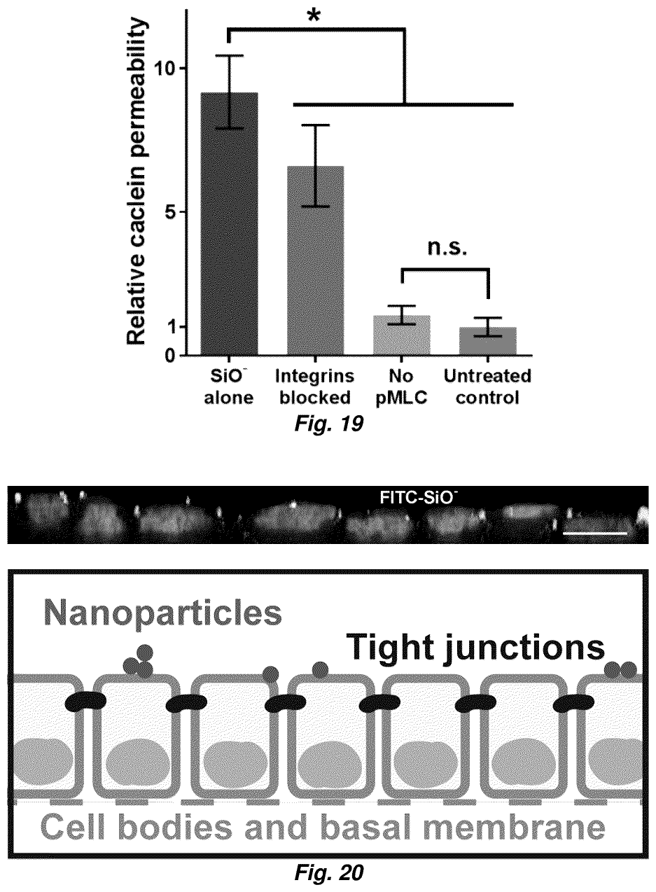

To accelerate clinical translation of this invention, the mechanism of action of anionic nanoparticles on intestinal cells was determined. Specifically, the potential for nanomaterials to bind epithelial cell surface receptors called integrins was investigated. The resulting signal cascade from the integrins activates the enzyme myosin light chain kinase (MLCK), which phosphorylates a portion of the cytoskeleton (MLC to pMLC). The cytoskeleton contracts and exerts tension on the tight junctions, causing them to open and allowing macromolecule drugs to diffuse into the body (FIG. 18). To determine whether this signaling pathway is also employed by the nanoparticles studied here, Caco-2 monolayers were treated to either block two integrins commonly associated with extracellular matrices, or to inhibit the action of MLCK. The particles' permeabilizing effects on these cells was then re-examined (FIG. 19). By preventing two of the twenty-four known human integrin subunits from binding nanoparticles, we reduced calcein permeability by approximately 30%. By contrast, treatment with an MLCK inhibitor completely prevented the particle treatments from increasing monolayer permeability. From this, it was concluded that silica nanoparticles likely interact with more integrin subtypes than examined here, but undoubtedly activate the MLCK-dependent pathway to rearrange tight junctions.

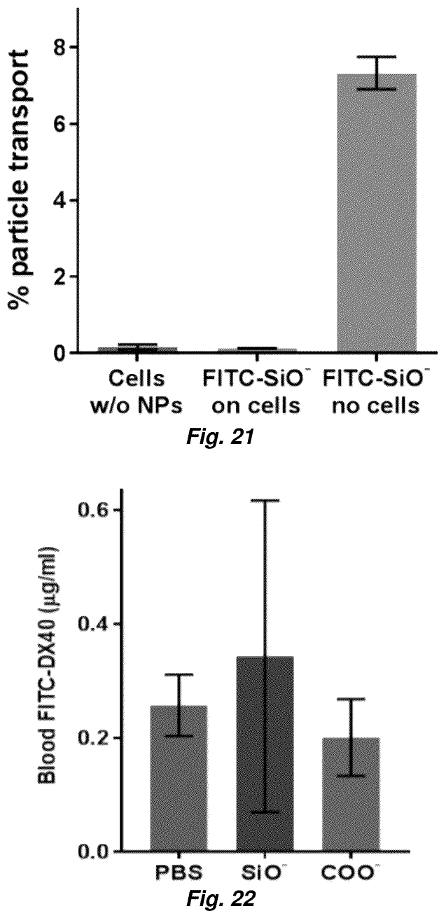

Next, it was demonstrated that the nanoparticles would not infiltrate and damage the epithelium. First, Caco-2 monolayers were treated with silica particles internally tagged with FITC, then imaged them using confocal microscopy. Assembling a composite stack of these monolayer images shows that the particles accumulate at the apical cell surface, but do not accumulate within junctions or inside the cells (FIG. 20). Additionally, the particles did not traverse the live monolayers during the three-hour course of the treatment period, with only the cell-free membrane supports allowing FITC-accumulation in the basal (bottom) wells (FIG. 21).

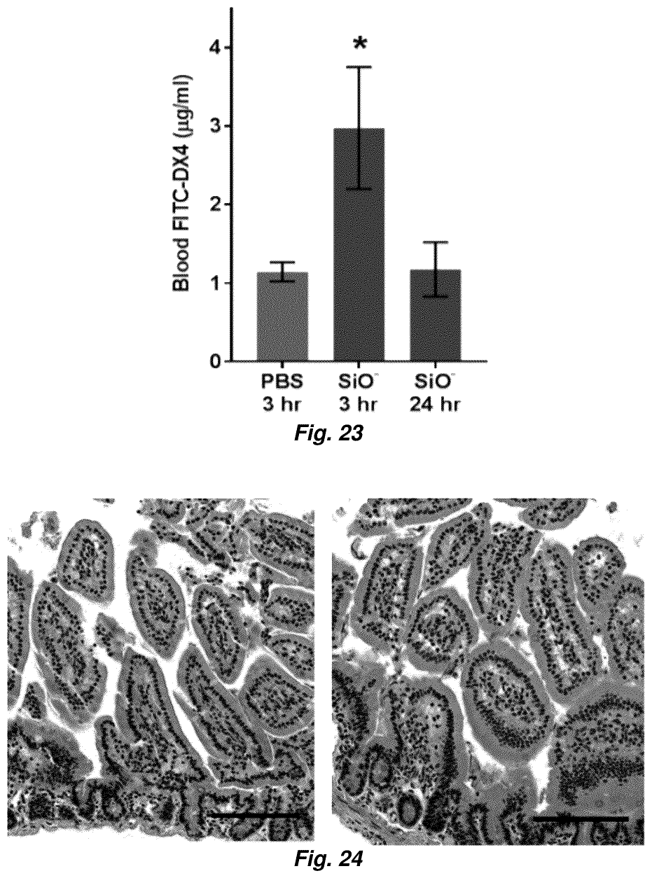

Safety aspects of particle-mediated permeation enhancement in mouse intestines were further examined. There was no significant difference exhibited in intestinal permeability to 40,000 MW, FITC-labelled dextran (FITC-DX40) between untreated and particle treated mice (FIG. 22). Since bacteria passing through intestinal epithelia are best modelled by molecules at least an order of magnitude larger than FITC-DX40, it is unlikely that the changes in permeability seen here would be sufficient to allow bacterial infiltration and subsequent inflammation of the intestinal wall. It was additionally established that particle induced permeability is reversible, as the intestinal absorption of FITC-DX4 in particle treated mice returned to low baseline levels when examined twenty-four hours after particle treatment (FIG. 23).

Finally, the intestines of untreated and particle-treated mice underwent histological analysis. There were no significant differences between the apparent tissue health of the control (FIG. 24, left) or experimental mice (FIG. 24, right). Further, semi-quantitative analysis by a trained pathologist indicated no difference in immune cell infiltration or inflammatory response between the samples. These results agree with work showing that feeding nanosilica to mice, even over extended periods of up to 10 weeks, does not impact their overall health.

These data provide compelling evidence that co-delivery with silica nanoparticles greatly boosts the activity of oral insulin, rivaling outcomes from the current gold standard of administration, subcutaneous injection. These results will enhanced and reinforced in the near future with nonhuman primate studies.

### Methods

### Cell Culture

Caco-2 cells were cultured in DMEM medium supplemented with 10% FBS, 1% Pen/Strep, and 0.1% Amphotericin B (“Caco-2 media”). Cells were passaged every 3 to 4 days at ratios between 1:3 and 1:8.

### Preparation of Particle Treatments

Treatments for cell culture studies were prepared by diluting suspensions into Caco-2 media (for MTT assays) or Enterocyte Differentiation Media (EDM, for TEER and permeability experiments) at the specified concentrations. Treatments for mouse studies utilized PBS to dilute particles to their designated concentrations.

### MTT Assay

Caco-2 cells were seeded in a clear, 96-well plate at a concentration of 10⁵ cells/well. After incubating the plate overnight at 37° C., the media in the wells was aspirated and replaced with the treatment solutions (100 μL/well). After three hours of exposure, the treatments were aspirated and the cells rinsed with warm PBS. MTT reagent (10 μL/well) and Caco-2 media (100 μL/well) were added to the wells. Three hours later, detergent reagent was added (100 μL/well) and the plate incubated at room temperature, overnight, in the dark. An automated plate reader was then used to measure the absorbance of the MTT product in each well. The viability of each treatment is expressed as the ratio of its wells' absorbance values to the absorbance values of untreated wells.

### Caco-2 Cell Monolayers

Caco-2 cells were suspended in Basal Seeding Medium (BSM) and seeded onto a collagen-coated, 24-Multiwell Insert Transwell® Plate at a concentration of 2×10⁵ cells per well. The plate was incubated at 37° C. for one to two days. On the third day, the BSM was replaced with Enterocyte Differentiation Medium (EDM). The plate was then incubated one to two more days at 37° C. to allow complete differentiation of monolayers.

### TEER Assay

All TEER (transepithelial electrical resistance) values were measured using a voltohmmeter. Between each TEER measurement, the plate was incubated at 37° C. for a minimum of fifteen minutes to allow the cells to rest. To ensure that all the wells contained fully formed Caco-2 monolayers (>200 Ω·cm²), initial TEER values were measured prior to treatment exposure. After adding the extracts to each well, TEER values were measured at defined intervals for a total of three hours.

### Permeability Assays

The paracellular diffusion markers were applied at 0.5 mM (calcein) or 0.1 mM (4 kDa FITC-Dextran), dissolved in EDM with the particle treatments, to the apical side of fully-formed monolayers (TEER>200 Ω·cm²). Fresh EDM with the relevant marker was used as a negative control. After one hour, media in the basal chambers was sampled and examined for fluorescence at 495/515 nm. Application of calibration curves yielded an amount of mass transferred across each monolayer, which was used in the permeability equation

$${P}_{app} = \frac{Δ M}{{C}_{a} A Δ t} ,$$

where Pₐₚₚ is the apparent permeability through the monolayer, ΔM is the marker mass in the basal compartment, Cₐ is the apical marker concentration, A is the monolayer area, and Δt is the time between samples. Permeability measurements are expressed as the ratio of monolayer permeability at 3 hours after treatment addition to permeability before treatment addition, normalized to any change in untreated control monolayers during that time.

### Confocal Microscopy

Monolayers were rinsed to remove treatments and fixed in ice cold methanol. They were next permeabilized with Triton-X100, then blocked with BSA solution to limit non-specific antibody binding, before being incubated for one hour with staining solutions. The staining solution contained DAPI to mark nucleic acids, AlexaFluor 488® conjugated Phalloidin to bind actin, and AlexaFluor® 594 conjugated Anti-ZO-1 antibodies. After staining, the monolayers were mounted on slides and imaged at 63× magnification using a Zeiss Laser Scanning Microscope.

### In Vitro Mucus Assay

Mucus was simulated by dissolving 5% (w/v) Type II porcine mucin in PBS, then applying to Transwell® permeable membrane supports (1 μm pore size) to give a 2 mm deep layer. The transwells were placed into a basal plate containing 1 ml of PBS in each well, and the particle suspensions added to the apical surface of the mucus. Samples were taken from the basal wells over time with PBS replenishment, and read on a plate reader to determine the fraction of particles transported across the barrier.

### Intestinal Permeability to Dextran

For dextran efficacy studies, fasted mice were orally gavaged with 100 mg/kg nanoparticle solutions, then gavaged two hours later with 600 mg/kg FITC-DX4. Three hours after the dextran gavage, blood was collected and centrifuged. The serum was removed and examined for FITC concentration by reading for fluorescence on the plate reader and comparing to a unique calibration curve for each experiment. For larger macromolecule studies, 40,000 MW dextran (FITC-DX40) was substituted at the same 600 mg/kg concentration. For permeability recovery, one group of mice was held for twenty-four hours, rather than two hours, between particle and FITC-DX4 gavages.

### Particle-Mucus Binding

Type II mucin was dissolved in water to a concentration of 10 mg/mL, stirring overnight at room temperature and sonicating to aid dissolution. The solution was then centrifuged for 30 minutes at 850×g to remove any undissolved solids. Nanoparticles were added to the mucin solution at 1 mg/mL particles, then kept at 37° C. with gentle stirring for the remainder of the experiment. At each time point, a sample of nanoparticle and mucin solution was collected and immediately examined for nanoparticle size via dynamic light scattering. Data shown are the averages of three DLS measurements on each sample.

### Intestinal Insulin Delivery

Following ten hours of fasting, mice were orally gavaged with PBS (for control) or nanoparticle suspensions (100 mg/kg unless otherwise specified). Two hours later, their initial blood sugar was measured, and the animals were placed under anesthesia. Their intestines were surgically exposed, and insulin was injected at the predetermined dose (1 unit per kg body weight unless otherwise specified) into the duodenum. The mice were closed and secured with tissue adhesive, then kept under anesthesia as their blood sugar levels were monitored each hour for five hours. For comparison to the current standard of insulin delivery, subcutaneous injections were given to additional mice, into the scruff on their necks. To determine specific insulin concentrations, blood samples were collected and separated via centrifugation. The serum was subjected to ELISA analysis for human insulin (LifeTechnologies®, Carlsbad, CA) per the instructions of the kit manufacturer. The ELISA kit exhibited reliable detection of the bovine insulin used herein.

### Oral Insulin and Exenatide Delivery With Capsules

Dry capsule contents for 675 U/kg insulin doses were produced by combining insulin, the protease inhibitor aprotinin, and inactive bovine serum albumin (BSA) filler at a 3:1:1 ratio in aqueous solution, then lyophilizing. Filler for negative control capsules contained just lyophilized aprotinin and BSA (0:1:4). 40 U/kg and 10 U/kg capsule filler was created by diluting the stronger insulin powder with the negative control powder. Size M capsules were filled with approximately 3 mg filler, and their exact weights recorded. Each capsule was then dip coated 3 times in a 7% (w/v in ethanol) solution of Eudragit® L100-55, drying completely under gentle airflow following each coat. The total dry weight of polymer added to each capsule ranged from 0.4 to 0.8 mg.

Following a ten-hour fasting period, large (>30 g) mice were orally gavaged with PBS (controls) or 100 mg/kg 50 nm silica nanoparticles, then orally administered capsules two hours later. Capsules were chosen so small variations in filler weight matched small variations in mouse weight, giving insulin doses within 10% of the reported dose. The capsules were immediately flushed into the stomach with an additional gavage of PBS or 100 mg/kg silica. Blood glucose was measured every two hours for a total of ten hours, and normalized to each mouse's reading before capsule administration.

Dry capsule contents for 1 mg/kg exenatide doses were produced by combining exenatide, the aprotinin, and FITC-DX4 filler at a 1:20:79 ratio in aqueous solution, then lyophilizing. Exenatide capsules were packed, coated, and administered as with insulin capsules. Blood was collected every two hours for a total of eight hours, and the serum separated via centrifugation. The serum was subjected to ELISA analysis for exenatide (Peninsula Laboratories, Can Carlos, CA) per the instructions of the kit manufacturer.

### Integrin Blockade and MLCK Inhibition

For the integrin blockade, Caco-2 monolayers were incubated for an hour before treatment with 1:10 diluted anti-integrin αV and 1:40 diluted (25 μg/mL) anti-integrin β1 antibodies. Particle treatments were added without removing the antibodies, and all changes in permeability were normalized to monolayers that were treated with the antibodies but no particles. For MLCK inhibition, the same procedure was followed, adding 0.33 mM (0.44 mg/mL) PIK to the cell media instead of the antibodies.

### Histology

Three particle treated and three untreated control mice were sacrificed following FITC-DX4 absorption experiments, and their small intestines were immediately surgically removed. The organs were fixed for 24 hours in 4% paraformaldehyde, then transferred to 70% ethanol for shipment to Mass Histology Services (Worcester, MA, USA). There, paraffin sections were prepared and stained with hematoxylin and eosin for histological examination. A semi-quantitative analysis of tissue health, inflammation, and immune cell infiltration for each specimen was also prepared by a certified veterinary pathologist.

### Nonhuman Primate Studies

Nonhuman primate (NHP) studies proposed for the immediate future center around confirming the efficacy of these nanoparticle treatments in a better model of the human GI tract. NHPs will be sedated and gavaged with 50 nm silica nanoparticle suspension. After 2-3 hours, they will then be administered capsules containing human insulin, which will be washed down with an additional dose of particle suspension. Blood glucose for each animal will be monitored each hour for a total of 12 hours. We expect to see a much more drastic induction of hypoglycemia (sustained drop in blood glucose levels) from animals that are treated with the silica nanoparticles, compared to animals that receive the capsules with saline gavages.

The following numbered clauses described various aspects of the invention.

Clause 1. A dosage form comprising negatively-charged nanoparticles having an average diameter, e.g., a Z average diameter as determined by dynamic light scattering, of less than 1 μm, 500 nm, less than 200 nm, or less than 100 nm, e.g., 50 nm or 20 nm, an active ingredient of less than 40 kDa, 25 kDa, or 10 kDa, or a hydrodynamic radius of 10 nm or less, and a pharmaceutically acceptable excipient.

Clause 2. The dosage form of clause 1, wherein the active ingredient is a peptide or a protein.

Clause 3. The dosage form of clause 1, wherein the active ingredient is insulin.

Clause 4. The dosage form of clause 1, wherein the active ingredient is a glucagon-like peptide-1 receptor agonist, such as exenatide; liraglutide; lixisenatide; albiglutide; dulaglutide; semaglutide; or taspoglutide.

Clause 5. The dosage form of clause 3, wherein the active ingredient is exenatide.

Clause 6. The dosage form of clause 1, wherein the nanoparticles are embedded in a biocompatible mucoadhesive.

Clause 7. The dosage form of clause 6, wherein the active ingredient is contained within a mucoadhesive.

Clause 8. The dosage form of clause 7, wherein the active ingredient is contained within the mucoadhesive in which the nanoparticles are embedded.

Clause 9. The dosage form of clause 6 or 7, wherein the mucoadhesive comprising the embedded nanoparticles forms a first layer, and the active ingredient is contained within a second layer over the first layer.

Clause 10. The dosage form of any one of clauses 6-9, further comprising an impermeable layer over the mucoadhesive containing the nanoparticles and the active ingredient, but not covering at least a portion of the mucoadhesive.

Clause 11. The dosage form of any one of clauses 1-10, wherein the nanoparticles are silica nanoparticles.

Clause 12. The dosage form of clause 11, wherein the silica particles are mesoporous and the active ingredient is contained in pores of the silica nanoparticles.

Clause 13. The dosage form of any one of clauses 1-12, wherein the ζ-potential of the nanoparticles ranges from less than 0 mV to −80 mV, from −20 mV to −80 mV, or from −30 mV to −50 mV.

Clause 14. The dosage form of any one of clauses 1-13, comprising a dissolvable coating layer containing the nanoparticles and the active ingredient.

Clause 15. The dosage form of clause 14, wherein the coating is an enteric coating.

Clause 16. A device, unit dosage form, or drug product, comprising the dosage form of any one of clauses 1-15.

Clause 17. A trans-epithelial drug delivery method, comprising: contacting an epithelial membrane with a negatively-charged nanoparticle having an average diameter, e.g., a Z average diameter as determined by dynamic light scattering, of less than 1 μm, 500 nm, less than 200 nm, or less than 100 nm, e.g., 50 nm or 20 nm; and then contacting the epithelial membrane with an active ingredient of less than 40 kDa, 25 kDa or less, or 10 kDa or less, or a hydrodynamic radius of 10 nm or less.

Clause 18. The method of clause 17, wherein the epithelial membrane is intestinal mucosa.

Clause 19. The method of clause 17, wherein the epithelial membrane is respiratory, pulmonary, vaginal, oral, intestinal, nasal, or urethral mucosal.

Clause 20. The method of clause 17, wherein the nanoparticles and active ingredient are administered in a single unit dosage form.

Clause 21. The method of clause 17, wherein the nanoparticles and the active ingredient are administered in separate unit dosage forms.

Clause 22. The method of any one of clauses 20 or 21 wherein the dosage form or dosage forms comprise a coating around the nanoparticles and the active ingredient.

Clause 23. The method of clause 22, wherein the coating is an enteric coating.

Clause 24. The method of any one of clauses 17-23, wherein the nanoparticles are administered embedded within a mucoadhesive.

Clause 25. The method of clause 24, wherein the active ingredient is mixed with a mucoadhesive.

Clause 26. The method of any one of clauses 17-25, wherein the nanoparticles are silica nanoparticles.

Clause 27. The method of clause 26, wherein the silica particles are mesoporous and the active ingredient is contained in pores of the silica nanoparticles.

Clause 28. The method of any one of clauses 17-27, wherein the ζ-potential of the nanoparticles ranges from less than 0 mV to −80 mV, from −20 mV to −80 mV, or from −30 mV to −50 mV.

Clause 29. The method of any one of clauses 17-28, wherein the active ingredient is a peptide or a protein.

Clause 30. The method of any one of clauses 17-28, wherein the active ingredient is insulin.

Clause 31. The method of any one of clauses 17-28, wherein the active ingredient is a glucagon-like peptide-1 receptor agonist, such as exenatide; liraglutide; lixisenatide; albiglutide; dulaglutide; semaglutide; or taspoglutide.

Clause 32. The method of any one of clauses 17-28, wherein the active ingredient is exenatide.

Clause 33. A method of treating diabetes, e.g., type 1 or type 2 diabetes, or insulin resistance, or inducing weight loss, in a patient, comprising administering to the patient an amount of insulin or a glucagon-like peptide-1 receptor agonist, such as exenatide; liraglutide; lixisenatide; albiglutide; dulaglutide; semaglutide; or taspoglutide, effective to treat diabetes or insulin resistance or to induce weight loss in a patient, thereby treating the diabetes or insulin resistance, or inducing weight loss, in the patient.

Clause 34. The method of clause 33, further comprising administering to the patient an amount of metformin, gliclazide, pioglitazone, repaglinide, acarbose, or a gliptin effective to treat diabetes or reduce liver glucose production in the patient.

The present invention has been described with reference to certain exemplary embodiments, dispersible compositions and uses thereof. However, it will be recognized by those of ordinary skill in the art that various substitutions, modifications or combinations of any of the exemplary embodiments may be made without departing from the spirit and scope of the invention. Thus, the invention is not limited by the description of the exemplary embodiments, but rather by the appended claims as originally filed.

What is claimed is:

1. A dosage form comprising:
negatively-charged nanoparticles having an average diameter of 200 nm or less; and
an active ingredient of less than 40 kDa or having a hydrodynamic radius of 10 nm or less, wherein the active ingredient is physically separate from the negatively-charged nanoparticles,
wherein the dosage form is configured such that the negatively-charged nanoparticles contact a patient's epithelial membrane before the active ingredient, thereby increasing permeability of the epithelial membrane.

2. The dosage form of claim 1, wherein the active ingredient is a peptide or a protein.

3. The dosage form of claim 1, wherein the active ingredient is insulin.

4. The dosage form of claim 1, wherein the active ingredient is a glucagon-like peptide-1 receptor agonist selected from exenatide, liraglutide, lixisenatide, albiglutide, dulaglutide, semaglutide, and taspoglutide.

5. The dosage form of claim 1, wherein the active ingredient is arranged in a first layer and the negatively-charged nanoparticles are arranged in a second, separate layer, the second layer configured to contact a patient's epithelium membrane before the first layer.

6. The dosage form of claim 1, wherein the active ingredient is arranged in a core and the negatively-charged nanoparticles are arranged in a layer that surrounds the core.

7. The dosage form of claim 1, wherein the negatively-charged nanoparticles are silica nanoparticles.

8. The dosage form of claim 1, wherein the ζ-potential of the negatively-charged nanoparticles ranges from less than 0 mV to −80 mV.

9. The dosage form of claim 1, further comprising a dissolvable enteric coating layer.

10. The dosage form of claim 1, wherein the negatively-charge nanoparticles are present in an amount of at least 0.05 wt. %.

11. A trans-epithelial drug delivery method, comprising:
contacting an epithelial membrane with negatively-charged nanoparticles having an average diameter of 200 nm or less, thereby increasing permeability of the epithelial membrane; and
then contacting the epithelial membrane with an active ingredient of less than 40 kDa, or having a hydrodynamic radius of 10 nm or less,
wherein the active ingredient is physically separate from the negatively-charged nanoparticles.

12. The method of claim 11, wherein the epithelial membrane is intestinal, respiratory, pulmonary, vaginal, oral, nasal, or urethral mucosal.

13. The method of claim 11, wherein the negatively-charged nanoparticles and the active ingredient are administered in a single unit dosage form.

14. The method of claim 11, wherein the negatively-charged nanoparticles and the active ingredient are administered in separate unit dosage forms.

15. The method of claim 11, wherein the active ingredient is mixed with a mucoadhesive.

16. The method of claim 11, wherein the negatively-charged nanoparticles are silica nanoparticles.

17. The method of claim 11, wherein the ζ-potential of the negatively-charged nanoparticles ranges from less than 0 mV to −80 mV.

18. The method of claim 11, wherein the active ingredient is a peptide or a protein.

19. The method of claim 11, wherein the active ingredient is insulin.

20. The method of claim 11, wherein the active ingredient is a glucagon-like peptide-1 receptor agonist selected from exenatide, liraglutide, lixisenatide, albiglutide, dulaglutide, semaglutide, and taspoglutide.


SEQUENCE LISTING (3 sequences)

SEQ ID NO: 1 - protein, 39 aa, Homo sapiens
HGEGTFTSDLSKQMEEEAVRLFIEWLKNGGPSSGAPPPS

SEQ ID NO: 2 - protein, 32 aa, Homo sapiens
CGNLSTCMLGTYTQDFNKFHTFPQTAIGVGAP

SEQ ID NO: 3 - protein, 32 aa, Oncorhynchus keta
CSNLSTCVLGKLSQELHKLQTYPRTNTGSGTP